SEQUENCE LISTING (35 sequences)

SEQ ID NO: 1 - DNA, 1638 bp, Synechococcus elongatus PCC7002
  misc_feature: Gene Nucleotide sequence of N1-ketoglutarate decarboxylase (Kgd)
atgaacaccgccgagctgctgattcgttgcctggaaaacgagggcgtggagtatatcttcggcttaccgggtgaagagaacctgcacatcctggaagcactgaaggaaagcccgatccgcttcattaccgttcgccatgagcagggtgccgcatttatggccgacgtttacggccgcttaaccggcaaggccggcgtttgcttaagtaccttaggccctggcgcaaccaacttaatgacaggcgttgccgacgccaacctggatggtgccccgttaatcgcaattaccggccaggttggtacagaccgcatgcacattgagagtcaccagtatctggacctggtggccatgttcgcacctgtgacaaagtggaataagcagatcgtgcgccctaacacaacacctgaggttgtgcgccgcgccttcaagatcgcacagcaggaaaagccgggcgccgttcatattgacctgccggagaatatcgccgccatgccggttgaaggccaacctttacagcgcgacggtcgcgagaagatctacgcaagcagtcgcagcctgaaccgtgccgccgaagccattgcacacgccaagagtccgctgatcctggttggcaacggcatcatccgcgcagacgcagcagaggccttaaccgacttcgccacccagctgaatatcccggtggtgaataccttcatgggcaagggcgccatcccttatacccacccgttaagcctgtggaccgtgggtctgcaacagcgcgactttgttacctgcgccttcgagcagagcgatctggttatcgcagtgggctacgacctgatcgagtacagcccgaaacgttggaatccggagggtaccaccccgatcatccatatcggcgaggtggccgcagagatcgatagcagttacatcccgctgacagaggttgtgggtgacatcggcgacgccttaaacgaaattcgcaagcgcaccgatcgcgaaggcaaaacagcaccgaaattcttaaacgtgcgcgccgagattcgcgaggattacgaacgccacggcaccgacgccagttttccggtgaagcctcagaagatcatctacgacctgcgccaagtgatggcccctgaagacatcgtgatcagcgacgtgggcgcccacaaaatgtggatggcacgccactaccactgcgaccgccctaacacctgtctgatcagtaacggctttgccgccatgggcatcgcaatccctggcgcagttgcagccaaactggtgtacccggagaagaacgtggtggccgttaccggtgatggtggcttcatgatgaactgccaggaactggagaccgccctgcgcattggcgcaaacttcgttaccctgatcttcaacgatggtggctacggcttaatcggctggaagcagatcaaccagttcggcgcacctgcatttgtggagtttggcaacccggacttcgttcaattcgccgagagcatgggcctgaaaggttaccgcattacagcagcagcagacctggttccgaccctgaaagaagcattagcccaggacgtgccggcagtgattgactgcccggttgactatagcgagaacgtgaagttcagccaaaagagcggcgacctgatctgtcgtatgtaa

SEQ ID NO: 2 - DNA, 1449 bp, Escherichia coli
  misc_feature: Gene Nucleotide sequence of succinate semialdehyde dehydrogenase (GabD)
atgaaacttaacgacagtaacttattccgccagcaggcgttgattaacggggaatggctggacgccaacaatggtgaagccatcgacgtcaccaatccggcgaacggcgacaagctgggtagcgtgccgaaaatgggcgcggatgaaacccgcgccgctatcgacgccgccaaccgcgccctgcccgcctggcgcgcgctcaccgccaaagaacgcgccaccattctgcgcaactggttcaatttgatgatggagcatcaggacgatttagcgcgcctgatgaccctcgaacagggtaaaccactggccgaagcgaaaggcgaaatcagctacgccgcctcctttattgagtggtttgccgaagaaggcaaacgcatttatggcgacaccattcctggtcatcaggccgataaacgcctgattgttatcaagcagccgattggcgtcaccgcggctatcacgccgtggaacttcccggcggcgatgattacccgcaaagccggtccggcgctggcagcaggctgcaccatggtgctgaagcccgccagtcagacgccgttctctgcgctggcgctggcggagctggcgatccgcgcgggcgttccggctggggtatttaacgtggtcaccggttcggcgggcgcggtcggtaacgaactgaccagtaacccgctggtgcgcaaactgtcgtttaccggttcgaccgaaattggccgccagttaatggaacagtgcgcgaaagacatcaagaaagtgtcgctggagctgggcggtaacgcgccgtttatcgtctttgacgatgccgacctcgacaaagccgtggaaggcgcgctggcctcgaaattccgcaacgccgggcaaacctgcgtctgcgccaaccgcctgtatgtgcaggacggcgtgtatgaccgttttgccgaaaaattgcagcaggcagtgagcaaactgcacatcggcgacgggctggataacggcgtcaccatcgggccgctgatcgatgaaaaagcggtagcaaaagtggaagagcatattgccgatgcgctggagaaaggcgcgcgcgtggtttgcggcggtaaagcgcacgaacgcggcggcaacttcttccagccgaccattctggtggacgttccggccaacgccaaagtgtcgaaagaagagacgttcggccccctcgccccgctgttccgctttaaagatgaagctgatgtgattgcgcaagccaatgacaccgagtttggccttgccgcctatttctacgcccgtgatttaagccgcgtcttccgcgtgggcgaagcgctggagtacggcatcgtcggcatcaataccggcattatttccaatgaagtggccccgttcggcggcatcaaagcctcgggtctgggtcgtgaaggttcgaagtatggcatcgaagattacttagaaatcaaatatatgtgcatcggtctttaa

SEQ ID NO: 3 - DNA, 1314 bp, Corynebacterium glutamicum
  misc_feature: Nucleotide sequence of citrate synthase (GltA)
atgtttgaaagggatatcgtggctactgataacaacaaggctgtcctgcactaccccggtggcgagttcgaaatggacatcatcgaggcttctgagggtaacaacggtgttgtcctgggcaagatgctgtctgagactggactgatcacttttgacccaggttatgtgagcactggctccaccgagtcgaagatcacctacatcgatggcgatgcgggaatcctgcgttaccgcggctatgacatcgctgatctggctgagaatgccaccttcaacgaggtttcttacctacttatcaacggtgagctaccaaccccagatgagcttcacaagtttaacgacgagattcgccaccacacccttctggacgaggacttcaagtcccagttcaacgtgttcccacgcgacgctcacccaatggcaaccttggcttcctcggttaacattttgtctacctactaccaggaccagctgaacccactcgatgaggcacagcttgataaggcaaccgttcgcctcatggcaaaggttccaatgctggctgcgtacgcacaccgcgcacgcaagggtgctccttacatgtacccagacaactccctcaatgcgcgtgagaacttcctgcgcatgatgttcggttacccaaccgagccatacgagatcgacccaatcatggtcaaggctctggacaagctgctcatcctgcacgctgaccacgagcagaactgctccacctccaccgttcgtatgatcggttccgcacaggccaacatgtttgtctccatcgctggtggcatcaacgctctgtccggcccactgcacggtggcgcaaaccaggctgttctggagatgctcgaagacatcaagagcaaccacggtggcgacgcaaccgagttcatgaacaaggtcaagaacaaggaagacggcgtccgcctcatgggcttcggacaccgcgtttacaagaactacgatccacgtgcagcaatcgtcaaggagaccgcacacgagatcctcgagcacctcggtggcgacgatcttctggatctggcaatcaagctggaagaaattgcactggctgatgattacttcatctcccgcaagctctacccgaacgtagacttctacaccggcctgatctaccgcgcaatgggcttcccaactgacttcttcaccgtattgttcgcaatcggtcgtctgccaggatggatcgctcactaccgcgagcagctcggtgcagcaggcaacaagatcaaccgcccacgccaggtctacaccggcaacgaatcccgcaagttggttcctcgcgaggagcgctaa

SEQ ID NO: 4 - DNA, 2760 bp, Corynebacterium glutamicum
  misc_feature: Gene Nucleotide sequence of phosphoenolpyruvate carboxylase (Ppc)
atgactgattttttacgcgatgacatcaggttcctcggtcaaatcctcggtgaggtaattgcggaacaagaaggccaggaggtttatgaactggtcgaacaagcgcgcctgacttcttttgatatcgccaagggcaacgccgaaatggatagcctggttcaggttttcgacggcattactccagccaaggcaacaccgattgctcgcgcattttcccacttcgctctgctggctaacctggcggaagacctctacgatgaagagcttcgtgaacaggctctcgatgcaggcgacacccctccggacagcactcttgatgccacctggctgaaactcaatgagggcaatgttggcgcagaagctgtggccgatgtgctgcgcaatgctgaggtggcgccggttctgactgcgcacccaactgagactcgccgccgcactgtttttgatgcgcaaaagtggatcaccacccacatgcgtgaacgccacgctttgcagtctgcggagcctaccgctcgtacgcaaagcaagttggatgagatcgagaagaacatccgccgtcgcatcaccattttgtggcagaccgcgttgattcgtgtggcccgcccacgtatcgaggacgagatcgaagtagggctgcgctactacaagctgagccttttggaagagattccacgtatcaaccgtgatgtggctgttgagcttcgtgagcgtttcggcgagggtgttcctttgaagcccgtggtcaagccaggttcctggattggtggagaccacgacggtaacccttatgtcaccgcggaaacagttgagtattccactcaccgcgctgcggaaaccgtgctcaagtactatgcacgccagctgcattccctcgagcatgagctcagcctgtcggaccgcatgaataaggtcaccccgcagctgcttgcgctggcagatgcagggcacaacgacgtgccaagccgcgtggatgagccttatcgacgcgccgtccatggcgttcgcggacgtatcctcgcgacgacggccgagctgatcggcgaggacgccgttgagggcgtgtggttcaaggtctttactccatacgcatctccggaagaattcttaaacgatgcgttgaccattgatcattctctgcgtgaatccaaggacgttctcattgccgatgatcgtttgtctgtgctgatttctgccatcgagagctttggattcaacctttacgcactggatctgcgccaaaactccgaaagctacgaggacgtcctcaccgagcttttcgaacgcgcccaagtcaccgcaaactaccgcgagctgtctgaagcagagaagcttgaggtgctgctgaaggaactgcgcagccctcgtccgctgatcccgcacggttcagatgaatacagcgaggtcaccgaccgcgagctcggcatcttccgcaccgcgtcggaggctgttaagaaattcgggccacggatggtgcctcactgcatcatctccatggcatcatcggtcaccgatgtgctcgagccgatggtgttgctcaaggaattcggactcatcgcagccaacggcgacaacccacgcggcaccgtcgatgtcatcccactgttcgaaaccatcgaagatctccaggccggcgccggaatcctcgacgaactgtggaaaattgatctctaccgcaactacctcctgcagcgcgacaacgtccaggaagtcatgctcggttactccgattccaacaaggatggcggatatttctccgcaaactgggcgctttacgacgcggaactgcagctcgtcgaactatgccgatcagccggggtcaagcttcgcctgttccacggccgtggtggcaccgtcggccgcggtggcggaccttcctacgacgcgattcttgcccagcccaggggggctgtccaaggttccgtgcgcatcaccgagcagggcgagatcatctccgctaagtacggcaaccccgaaaccgcgcgccgaaacctcgaagccctggtctcagccacgcttgaggcatcgcttctcgacgtctccgaactcaccgatcaccaacgcgcgtacgacatcatgagtgagatctctgagctcagcttgaagaagtacgcctccttggtgcacgaggatcaaggcttcatcgattacttcacccagtccacgccgctgcaggagattggatccctcaacatcggatccaggccttcctcacgcaagcagacctcctcggtggaagatttgcgagccatcccatgggtgctcagctggtcacagtctcgtgtcatgctgccaggctggtttggtgtcggaaccgcattagagcagtggattggcgaaggggagcaggccacccaacgcattgccgagctgcaaacactcaatgagtcctggccatttttcacctcagtgttggataacatggctcaggtgatgtccaaggcagagctgcgtttggcaaagctctacgcagacctgatcccagatacggaagtagccgagcgagtctattccgtcatccgcgaggagtacttcctgaccaagaagatgttctgcgtaatcaccggctctgatgatctgcttgatgacaacccacttctcgcacgctctgtccagcgccgatacccctacctgcttccactcaacgtgatccaggtagagatgatgcgacgctaccgaaaaggcgaccaaagcgagcaagtgtcccgcaacattcagctgaccatgaacggtctttccactgcgctgcgcaactccggctaa

SEQ ID NO: 5 - DNA, 520 bp, Artificial sequence
  Synthetic   CDS - The sgRNA fragment binding dCas9 protein for guiding to the target intended to be inhibited, where the 20 base pairs N listed represent the corresponding gene fragment for inhibition, which varies depending on the target.
  misc_feature (227)..(246): n is a, c, g, or t
cccgggtaatctagacgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggannnnnnnnnnnnnnnnnnnngttttagagctagaaatagcaagttaaaataaggctagtccgttatcaacttgaaaaagtggcaccgagtcggtgcttttttttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaaactagt

SEQ ID NO: 6 - DNA, 20 bp, Artificial Sequence
  Synthetic sdhA-1
gctgccaagcccctgccaga

SEQ ID NO: 7 - DNA, 20 bp, Artificial Sequence
  Synthetic sdhA-2
cgccctgagccgccacgcta

SEQ ID NO: 8 - DNA, 20 bp, Artificial Sequence
  Synthetic sdhB-1
agatcgtgactgcaggaata

SEQ ID NO: 9 - DNA, 20 bp, Artificial Sequence
  Synthetic sdhB-2
ggaggcttgctgcaacgcat

SEQ ID NO: 10 - DNA, 20 bp, Artificial Sequence
  Synthetic glgC-1
ttggcgcgctgtttggttag

SEQ ID NO: 11 - DNA, 20 bp, Artificial Sequence
  Synthetic glgC-2
tcaagcggtatttgcccgcc

SEQ ID NO: 12 - DNA, 20 bp, Artificial Sequence
  Synthetic maeA-1
gaaacgctgtgactggcatt

SEQ ID NO: 13 - DNA, 20 bp, Artificial Sequence
  Synthetic maeA-2
gctgatcgtcgtgaaacacc

SEQ ID NO: 14 - DNA, 20 bp, Artificial Sequence
  Synthetic aspC-1
gcgatcgcgagagtcaacga

SEQ ID NO: 15 - DNA, 20 bp, Artificial Sequence
  Synthetic aspC-2
tcgggatagctcaaccagta

SEQ ID NO: 16 - DNA, 20 bp, Artificial Sequence
  Synthetic glsF-1
cagcgtgggccttggtaggt

SEQ ID NO: 17 - DNA, 20 bp, Artificial Sequence
  Synthetic glsF-2
tccccaagccaacgctcgac

SEQ ID NO: 18 - DNA, 20 bp, Artificial Sequence
  Synthetic zwf-1
gttcagggactttgtcttgg

SEQ ID NO: 19 - DNA, 20 bp, Artificial Sequence
  Synthetic zwf-2
ctggcggcaaacgccgttcg

SEQ ID NO: 20 - DNA, 20 bp, Artificial Sequence
  Synthetic pgi-1
gatcgtagtagagccaatcg

SEQ ID NO: 21 - DNA, 20 bp, Artificial Sequence
  Synthetic pgi-2
catccgctgttcatcggggt

SEQ ID NO: 22 - DNA, 20 bp, Artificial Sequence
  Synthetic spsA-1
ctgccctcgcagcagaccat

SEQ ID NO: 23 - DNA, 20 bp, Artificial Sequence
  Synthetic spsA-2
gacttgtggggatttagctt

SEQ ID NO: 24 - DNA, 20 bp, Artificial Sequence
  Synthetic pk-1
ggacgttgcagggccaatcg

SEQ ID NO: 25 - DNA, 20 bp, Artificial Sequence
  Synthetic pk-2
atgatcgtcgtgggttccgt

SEQ ID NO: 26 - DNA, 20 bp, Artificial Sequence
  Synthetic acsA-1
gaaaaacccgcttctcttgg

SEQ ID NO: 27 - DNA, 20 bp, Artificial Sequence
  Synthetic acsA-2
cagtccagcgtctgttgcca

SEQ ID NO: 28 - DNA, 20 bp, Artificial Sequence
  Synthetic pdhA-1
gcctgagaagcctgaaaact

SEQ ID NO: 29 - DNA, 20 bp, Artificial Sequence
  Synthetic pdhA-2
catggccttgataatgccgc

SEQ ID NO: 30 - DNA, 20 bp, Artificial Sequence
  Synthetic pdhB-1
ttcatcgattgcagcccgta

SEQ ID NO: 31 - DNA, 20 bp, Artificial Sequence
  Synthetic pdhB-2
accgaaagtcgccgtacttt

SEQ ID NO: 32 - DNA, 20 bp, Artificial Sequence
  Synthetic oxdC-1
actacctatcaggacaaaac

SEQ ID NO: 33 - DNA, 20 bp, Artificial Sequence
  Synthetic oxdC-2
ccaccgtcgtagtccacaag

SEQ ID NO: 34 - DNA, 20 bp, Artificial Sequence
  Synthetic opcA-1
tcagccagcgcgattggcgt

SEQ ID NO: 35 - DNA, 20 bp, Artificial Sequence
  Synthetic opcA-2
ttcatagaccacgatgctga

(19) United States
(12) Patent Application Publication
(10) Pub. No.: US 2020/0270647 A1
(43) Pub. Date: Aug. 27, 2020

(54) TRANSGENIC STRAIN FOR PRODUCING SUCCINATE AND METHOD FOR PRODUCING SUCCINATE

(71) Applicant: NATIONAL CHIAO TUNG UNIVERSITY, Hsinchu (TW)

(72) Inventors: Ethan I LAN, Hsinchu (TW); Martin LAI, Hsinchu (TW); Ryan TUNG, Hsinchu (TW); Jemmy TSAI, Hsinchu (TW); Jason KU, Hsinchu (TW)

(73) Assignee: NATIONAL CHIAO TUNG UNIVERSITY, Hsinchu (TW)

(21) Appl. No.: 16/797,510

(22) Filed: Feb. 21, 2020

(30) Foreign Application Priority Data
Feb. 22, 2019 (TW) 108106113

(51) Int. Cl. C12P 7/46; C12N 15/52

(52) U.S. Cl. CPC C12P 7/46; C12N 15/52; C12N 2310/20; C12Y 402/01082; C12Y 301/01; C12Y 302/01008; C12Y 101/01175

(57) ABSTRACT

The present invention provides a transgenic strain for producing succinate. The transgenic strain comprises an autotrophic host cell, and a plurality of exogenous genes within the host cell. The exogenous genes include a gene for expressing α-ketoglutarate decarboxylase (Kgd), a gene for expressing succinate semialdehyde dehydrogenase (GabD), a gene for expressing citrate synthase (GltA) and a gene for expressing phosphoenolpyruvate carboxylase (Ppc). Further, expression of at least one of the native genes encoding glucose-1-phosphate adenylyltransferase (GlgC), succinate dehydrogenase subunit A (SdhA), and succinate dehydrogenase subunit B (SdhB) is suppressed in the host cell. The present invention further provides a method for producing succinate, which comprises: providing a transgenic strain of the present invention, and culturing the transgenic strain under a preset condition.

### REFERENCE TO SEQUENCE LISTING SUBMITTED VIA EFS-WEB

This application includes an electronically submitted sequence listing in .txt format. The .txt file contains a sequence listing entitled “5199-0299PUS1_ST25.txt” created on Apr. 3, 2020 and is 17,324 bytes in size. The sequence listing contained in this .txt file is part of the specification and is hereby incorporated by reference herein in its entirety.

### BACKGROUND

### Technical Field

The present invention relates to a transgenic strain for producing succinate and a method for producing succinate. Specifically, the present invention relates to a transgenic strain capable of producing succinate under the condition of providing carbon dioxide and light, and a method for producing succinate with the transgenic strain.

### Related Art

Succinate is currently one of the most important industrial chemicals and is commonly used as a food additive and pharmaceutical material. In addition, succinate can also be used as a raw material to produce other important chemical derivatives, such as 1,4-butanediol, butadiene, tetrahydrofuran and the like. At present, succinate is mainly produced by chemical processes such as hydrogenation of maleic acid or maleic anhydride. Such chemical processes for the production of succinate often require petrochemical raw materials, thus substantially increasing the cost and dependence on petrochemical energy.

In order to reduce the dependence on petrochemical energy in the production of industrial raw materials, there is a need to develop a method for producing succinate without consuming petrochemical raw materials. In view of this, in the prior art, some heterotrophic organisms such as E. coli are developed for the production of succinate. However, these processes still require the supply of carbohydrates or other organic carbon sources such as glycerol to the heterotrophic organisms for producing succinate.

### SUMMARY

### Technical Means for Solving the Problems

To solve the above problems, an aspect of the present invention provides a transgenic strain for producing succinate. The transgenic strain comprises an autotrophic host cell, and a plurality of exogenous genes within the host cell. The exogenous genes comprise a first exogenous gene, which is a gene encoding α-ketoglutarate decarboxylase (Kgd), or a gene having 80% or higher sequence identity to SEQ ID NO: 1 and having Kgd activity; a second exogenous gene, which is a gene encoding succinate semialdehyde dehydrogenase (GabD), or a gene having 80% or higher sequence identity to SEQ ID NO: 2 and having GabD activity; a third exogenous gene, which is a gene encoding citrate synthase (GltA), or a gene having 80% or higher sequence identity to SEQ ID NO: 3 and having GltA activity; and a fourth exogenous gene, which is a gene encoding phosphoenolpyruvate carboxylase (Ppc), or a gene having 80% or higher sequence identity to SEQ ID NO: 4 and having Ppc activity. Expression of at least one of the native genes encoding glucose-1-phosphate adenylyltransferase (GlgC), succinate dehydrogenase subunit A (SdhA), and succinate dehydrogenase subunit B (SdhB) is suppressed in the host cell.

According to an embodiment of the present invention, expressions of the native genes encoding GlgC and SdhB are both suppressed in the host cell.

According to another embodiment of the present invention, the transgenic strain further comprises a recombinant plasmid in the host cell, and the recombinant plasmid comprises the first exogenous gene, the second exogenous gene, the third exogenous gene, and the fourth exogenous gene.

According to a further embodiment of the present invention, the first exogenous gene, the second exogenous gene, the third exogenous gene, and the fourth exogenous gene are introduced into the genome of the host cell.

According to another embodiment of the present invention, the host cell is selected from the group consisting of Synechococcus elongatus PCC7942, Synechococcus elongatus UTEX 2973, Synechocystis sp. PCC6803, and Synechococcus sp. PCC7002.

According to still another embodiment of the present invention, the host cell is a cyanobacterium comprising native genes encoding GlgC, SdhA, and SdhB, and capable of producing phosphoenolpyruvate with carbon dioxide.

According to a further embodiment of the present invention, the transgenic strain is capable of consuming carbon dioxide to produce succinate under the condition of providing carbon dioxide and light, and excreting the produced succinate out of the host cell.

According to another embodiment of the present invention, knockdown of at least one of the genes encoding GlgC, SdhA, and SdhB in the transgenic strain is performed by CRISPRi for gene suppression.

According to still another embodiment of the present invention, the exogenous genes further include a gene encoding a succinate transport protein.

According to a further embodiment of the present invention, the exogenous genes further include genes encoding D-xylose-proton symporter (xylE), D-xylose dehydrogenase (xylB), D-xylono-1,5-lactone lactonase (xylC), one of xylonate dehydratase (yjhG) or xylanase D (xylD), 2-keto-3-deoxyxylonate dehydratase (xylX), and α-ketoglutaric semialdehyde dehydrogenase (xylA).

According to another embodiment of the present invention, the transgenic strain encoding xylE, xylB, xylC, one of yjhG or xylD, xylX, and xylA can consume xylose to produce succinate in the presence of xylose, and excrete the produced succinate out of the host cell.

Another aspect of the present invention provides a method for producing succinate, which comprises: a) providing the transgenic strain transfected with the first to fourth exogenous genes and inhibiting the expression of at least one of the genes encoding GlgC, SdhA, and SdhB; and b) culturing the transgenic strain under a first preset condition. The first preset condition includes providing light and providing carbon dioxide.

A further aspect of the present invention provides a method for producing succinate, which comprises: a) providing the transgenic strain transfected with the first to fourth exogenous genes and the genes encoding xylE, xylB, xylC, one of yjhG or xylD, xylX and xylA, and inhibiting the expression of at least one of the genes encoding GlgC, SdhA, and SdhB; and b) culturing the transgenic strain under a first preset condition, a second preset condition, and a combination thereof. The first preset condition includes providing light and providing carbon dioxide; and the second preset condition includes proving xylose.

According to an embodiment of the present invention, the culturing of the transgenic strain under a first preset condition, a second preset condition, and a combination thereof comprises performing the first preset condition and the second preset condition alternately.

According to still another embodiment of the present invention, the first preset condition includes providing light at a light intensity of 100-800 uE.

According to a further embodiment of the present invention, the first preset condition includes providing light at a light intensity of 200 uE.

According to another embodiment of the present invention, the first preset condition further comprises: introducing mixed air having a gas composition comprising 0 to 5% carbon dioxide at a flow rate of 20 to 80 cc/min to a culture medium in which the transgenic strain is cultured at a culture temperature of 30° C.

### Technical Effect over Prior Art

According to the transgenic strain for producing succinate and the method for producing succinate provided in the present invention, succinate can be produced by consuming carbon dioxide under the condition of providing carbon dioxide and light. Therefore, succinate can be produced with carbon dioxide which has low cost and simple structure with respect to common petrochemical raw materials, thereby reducing the production cost of succinate and improving the dependence on petrochemical energy. In addition, according to the present invention, if succinate is produced by providing carbon dioxide, it is also possible to consume carbon dioxide which is excessively discharged in life or industry, thereby improving the environmental impact and harm (for example, greenhouse effect) caused by excessive carbon dioxide.

### BRIEF DESCRIPTION OF THE DRAWINGS

The patent or application file contains at least one color drawing. Copies of this patent or patent application publication with color drawing will be provided by the USPTO upon request and payment of the necessary fee.

FIG. 1 is a schematic diagram showing a succinate production pathway integrated into a native metabolic pathway in a host cell according to an embodiment of the present invention.

FIG. 2 is a schematic diagram showing the suppression of a native gene correlating or competing with a succinate production pathway in a host cell according to another embodiment of the present invention.

FIG. 3 is a schematic diagram showing the transport of succinate by a succinate transport protein encoded by an exogenous gene according to still another embodiment of the present invention.

FIG. 4 is a schematic diagram showing a xylose metabolic pathway integrated into a succinate production pathway according to a further embodiment of the present invention.

FIG. 5 is a schematic diagram showing the introduction of an exogenous gene into the chromosome of a host cell by constructing a plasmid according to an embodiment of the present invention. Panel A of FIG. 5 is a schematic diagram showing the insertion of a corresponding gene in the succinate production pathway into the NSI site of Synechococcus elongatus PCC7942; and Panel B of FIG. 5 is a schematic diagram showing the insertion of a sgRNA guide fragment of a corresponding gene to be knocked down by CRISPRi for gene suppression into the NSII site of Synechococcus elongatus PCC7942.

FIG. 6 is a schematic diagram showing the introduction of an exogenous gene into the chromosome of a host cell by constructing a plasmid according to another embodiment of the present invention. Panel A of FIG. 6 is a schematic diagram showing the insertion of a corresponding gene in the succinate production pathway into the NSI site of Synechococcus elongatus PCC7942; and Panel B of FIG. 6 is a schematic diagram showing the insertion of a corresponding gene in the xylose metabolic pathway into the NSII site of Synechococcus elongatus PCC7942.

FIG. 7 is a schematic diagram showing the introduction of an exogenous gene into a host cell by constructing a plasmid according to still another embodiment of the present invention. Panel A of FIG. 7 is a schematic diagram showing the insertion of a corresponding gene in the succinate production pathway into the NSI site of Synechococcus elongatus UTEX2973; and Panel B of FIG. 7 is a schematic diagram showing the introduction of a constructed plasmid carrying a corresponding gene in the succinate production pathway into the host cell as a cytoplasmic plasmid (Episomal).

FIG. 8 is a schematic diagram showing the cell density of and succinate consumption by Synechococcus elongatus PCC7942 cultured with different concentrations of disodium succinate or sodium chloride according to a further embodiment of the present invention. Panel A of FIG. 8 is a curve showing the cell density of Synechococcus elongatus PCC7942 as a function of days of culture; and Panel B of FIG. 8 is a bar graph showing the consumption of provided succinate by Synechococcus elongatus PCC7942 with days of culture.

FIG. 9 is a schematic diagram showing the cell density of and succinate production by an LAN1 strain transfected with GabD and Kgd, an LAN2 strain transfected with GabD, Kgd, and GltA, and an LAN3 strain transfected with Kgd, GabD, GltA, and Ppc as a function of days of culture according to an embodiment of the present invention. Panel A of FIG. 9 is a curve showing the concentration of succinate produced by LAN1, LAN2 and LAN3; Panel B of FIG. 9 is a curve showing the cell density of LAN1, LAN2 and LAN3; and Panel C of FIG. 9 is a bar graph showing the production rate of succinate produced by LAN1, LAN2 and LAN3.

FIG. 10 is a schematic diagram showing the cell density of and succinate production by the LAN3 strain transfected with Kgd, GabD, GltA, and Ppc and strains in which different native genes are further suppressed on the basis of LAN3 as a function of days of culture according to another embodiment of the present invention. Panel A of FIG. 10 is a curve showing the concentration of succinate produced by each strain; and Panel B of FIG. 10 is a curve showing the cell density of each strain.

FIG. 11 is a bar graph showing the succinate production by strains which are further transfected with an exogenous gene encoding the succinate transport protein sucE, dcuB, or dcuC on the basis of the LAN3 strain according to a further embodiment of the present invention.

FIG. 12 is a schematic diagram showing the cell density of and succinate production by the LAN3 strain and wild-type (WT) Synechococcus elongatus PCC7942 incubated alternately under light and in the dark according to another embodiment of the present invention. Panel A of FIG. 12 is a curve showing the cell density of each strain; and Panel B of FIG. 12 is a curve showing the concentration of succinate produced by each strain.

FIG. 13 is a schematic diagram showing the cell density of and succinate production by the wild-type (WT) Synechococcus elongatus PCC7942, the LAN3 strain, the DN01 strain and the DNO2 strain incubated alternately under light and in the dark according to still another embodiment of the present invention. Panel A of FIG. 13 is a curve showing the concentration of succinate produced by each strain with or without xylose; Panel B of FIG. 13 is a curve showing the cell density of each strain with or without xylose; and Panel C of FIG. 13 shows the residual amount of xylose over time with the culture of each strain.

FIG. 14 is a schematic diagram showing the cell density of and succinate production by a strain which is transfected with an exogenous gene inserted into the genome or as a cytoplasmic plasmid (episomal) according to an embodiment of the present invention. Panel A of FIG. 14 is a curve showing the cell density of each strain; and Panel B of FIG. 14 is a curve showing the concentration of succinate produced by each strain.

FIG. 15 is a schematic diagram showing the succinate production by the LAN3 strain after 12 days of irradiation at different light intensities according to another embodiment of the present invention.

FIG. 16 is a curve showing the concentration of succinate produced by the LAN3 strain and the strain in which GlgC and SdhB are suppressed on the basis of the LAN3 strain under light of 200 μE according to still another embodiment of the present invention.

### DETAILED DESCRIPTION

Various embodiments will be described below, and the spirit and principles of the present invention will be readily understood by those of ordinary skill in the art from reading the disclosure with reference to accompanying drawings. However, although some specific embodiments are particularly described herein, these embodiments are only to be considered as illustrative and not restrictive or exhaustive in any aspect. Therefore, various changes and modifications can be made to the present invention by those of ordinary skill in the art without departing from the spirit and scope of the present invention, which are apparent and can be easily achieved.

Succinate is an intermediate product in the Krebs cycle, also known as the tricarboxylic acid cycle or citric acid cycle, or a possible fermentation product in nature, which is generally not significantly toxic to organisms. In general, heterotrophic organisms need to use the Krebs cycle to break down substances such as carbohydrates to obtain the energy needed for growth and metabolism, and to synthesize some of the required chemicals. In contrast, autotrophic organisms generally utilize light energy directly and have the ability to convert carbon dioxide to carbohydrates using light energy, and thus do not require or have only incomplete Krebs cycle. For example, Synechococcus elongatus PCC7942 does not have a metabolic pathway for producing succinate, but can convert carbon dioxide into 3-phosphoglycerate by the Calvin cycle. In view of this, the present invention attempts to construct a path for converting carbon dioxide into succinate in an autotrophic strain, to replace the currently common chemical process or fermentation process, so as to produce succinate more efficiently. However, the metabolic pathway of a genetically modified strain has many hazards and may cause metabolic disorders, slowed or ceased growth, pathological changes, mutations, abnormal accumulation of metabolites, and the like, and even death to the host cell of the strain, Furthermore, even if genetic modification can be successfully utilized to produce the desired material, there is still a need to further overcome the challenge of extracting the desired material from the engineered strain.

According to various embodiments of the present invention, a genetic modification method is successfully used to produce a transgenic strain that can convert carbon dioxide into succinate under the condition of providing carbon dioxide and light, whereby succinate is produced by using carbon dioxide and light. Further, according to the present invention, the constructed transgenic strain can sustain the growth and metabolism, and no significant adverse effects on the host cells are observed. In addition, the transgenic strain constructed according to various embodiments of the present invention can excrete the produced succinate out of the host cell while culturing and growing. Therefore, according to the transgenic strain and the method for producing succinate of the present invention, the succinate can be produced, excreted and harvested while the transgenic strain is cultured, without additionally cell disruption/cell lysis or use of any method for extracting succinate from the host cell of the transgenic strain. This also avoids or reduces the possible harm and negative effects caused by excessive succinate accumulation in the host cell, and greatly simplifies and improves the process of extracting succinate.

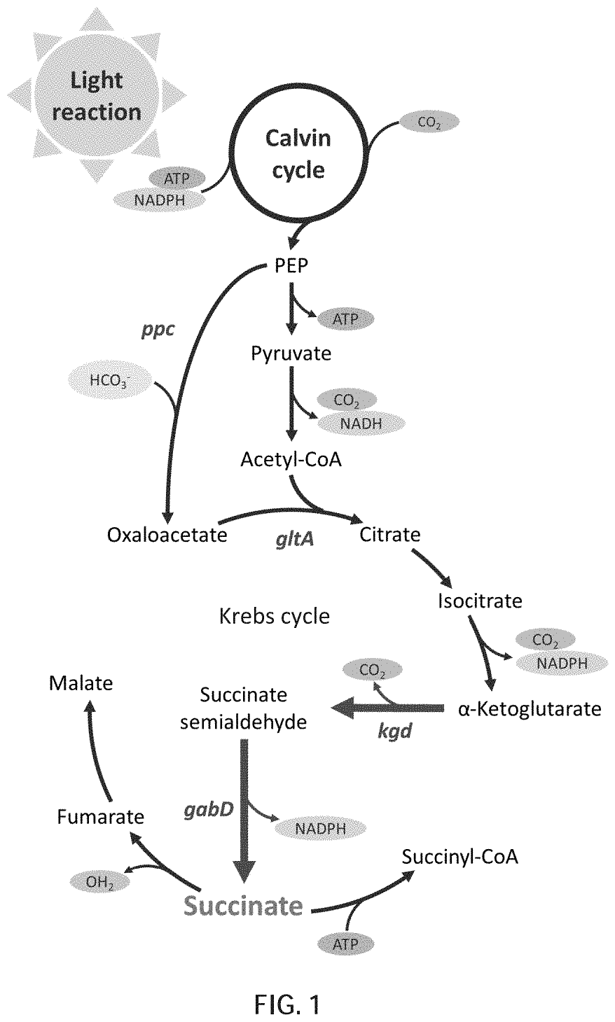

In particular, the cyanobacterium Synechococcus elongatus PCC7942 (hereinafter referred to as PCC7942) which does not have the Krebs cycle has a metabolic pathway for synthesizing α-ketoglutarate with carbon dioxide by the Calvin cycle through a number of processes in sequence. Based on this pathway, referring to FIG. 1, according to an embodiment of the present invention, a plurality of exogenous genes is introduced and expressed in the host cell of PCC7942, thereby constructing a succinate production pathway for synthesizing succinate from carbon dioxide. Specifically, as indicated by the red word in FIG. 1, the exogenous genes introduced into PCC7942 by genetic engineering comprise a gene encoding α-ketoglutarate decarboxylase (Kgd), or a gene having 80% or higher sequence identity to SEQ ID NO: 1 and having Kgd activity; a gene encoding succinate semialdehyde dehydrogenase (GabD), or a gene having 80% or higher sequence identity to SEQ ID NO: 2 and having GabD activity; a gene encoding citrate synthase (GltA), or a gene having 80% or higher sequence identity to SEQ ID NO: 3 and having GltA activity; and a gene encoding phosphoenolpyruvate carboxylase (Ppc), or a gene having 80% or higher sequence identity to SEQ ID NO: 4 and having Ppc activity. Therefore, the transfected transgenic strain can express α-ketoglutarate decarboxylase (Kgd), succinate semialdehyde dehydrogenase (GabD), citrate synthase (GltA), and phosphoenolpyruvate carboxylase (Ppc), or express proteins having α-ketoglutarate decarboxylase (Kgd), succinate semialdehyde dehydrogenase (GabD), citrate synthase (GltA), and phosphoenolpyruvate carboxylase (Ppc) activity. As shown in FIG. 1, the genetically modified transgenic strain can convert α-ketoglutarate to succinate semialdehyde and then to succinate through the previous pathway for α-ketoglutarate synthesis by using these enzymes or proteins.

In FIG. 1, the dashed arrow indicates the metabolic synthesis direction of the Krebs cycle in the original common heterotrophic organism. That is, a conventional heterotrophic organism causes glucose or a similar carbohydrate to undergo glycolysis to form pyruvate, and then pyruvate enters the Krebs cycle and is converted into α-ketoglutarate by the process shown in FIG. 1. Then, α-ketoglutarate is converted into succinyl CoA and then into succinate. Succinate continues the Krebs cycle, and is reduced by several procedures to produce oxaloacetate to complete the Krebs cycle. This process is well known to those of ordinary skill in the art and will not be described in detail herein. In contrast, the cyanobacterium strain PCC7942 according to an embodiment of the present invention does not have and does not use the same pathway of Krebs cycle as that in the heterotrophic organism to synthesize succinate, but alternatively develops a pathway to synthesize succinate sequentially from α-ketoglutarate, succinate semialdehyde to succinate.

Specifically, in order to allow the exogenous genes to be smoothly expressed in the cyanobacterium strain PCC7942, a host cell produced by transforming PCC7942 with the unique Kgd gene derived from Synechococcus elongatus PCC7002 that is also a cyanobacterium strain and the gabD gene derived from Escherichia coli is used in an embodiment of the present invention. In this way, it is possible to reduce the transfection and expression barriers that may be encountered in constructing a similar cycle to the Krebs cycle in a heterotrophic organism exclusively with the genes of a heterotrophic organism. However, with the same enzyme or protein function, the Kgd gene or the gabD gene from other species may also be used according to other embodiments, and the present invention is not limited thereto.

Further, according to an embodiment of the present invention, by simply expressing α-ketoglutarate decarboxylase (Kgd) and succinate semialdehyde dehydrogenase (GabD), succinate can be produced, but the growth of the transgenic strain of cyanobacterium is obviously retarded. This is most likely due to the excessive consumption of α-ketoglutarate, making the overall metabolism of the host cell imbalanced. This is not conducive to the sustainable production of succinate, and causes difficulty to high-throughput and high-efficiency production. In order to solve this problem, according to another embodiment of the present invention, an exogenous gene from Corynebacterium glutamicum can be further transfected to enhance the expression of citrate synthase (GltA) and phosphoenolpyruvate carboxylase (Ppc), thereby improving the above problem. As shown in FIG. 1, the citrate synthase (GltA) and phosphoenolpyruvate carboxylase (Ppc) are enzymes originally existing in PCC7942, so the introduction and expression of the exogenous genes encoding these two enzymes do not create any new pathway. However, by transferring the two exogenous genes and enhancing the expression of citrate synthase (GltA) and phosphoenolpyruvate carboxylase (Ppc), the balanced metabolism and growth of the host cells are promoted. Therefore, a balance is achieved between the production of succinate and the maintenance of the sustainable growth of the host cell.

According to some embodiments of the present invention, the method for producing a transgenic strain comprises transforming a recombinant plasmid comprising the exogenous genes encoding α-ketoglutarate decarboxylase (Kgd), succinate semialdehyde dehydrogenase (GabD), citrate synthase (GltA), and phosphoenolpyruvate carboxylase (Ppc) into the host cell, and allowing the recombinant plasmid independent of the genome of the host cell and located in the host cell to express and/or replicate. In addition, according to some other embodiments of the present invention, the method for producing a transgenic strain comprises transforming a recombinant plasmid comprising the exogenous gene encoding Kgd, GabD, GltA and Ppc into the host cell, and subjecting the recombinant plasmid to, for example, homologous recombination with the genome of the host cell, to introduce the exogenous genes into the genome of the host cell for expression. However, the present invention is not limited thereto. Various conventional or future developed techniques can be utilized to feed the four exogenous genes in the host cell or interpose the four exogenous genes into the genome of the host cell for expression.

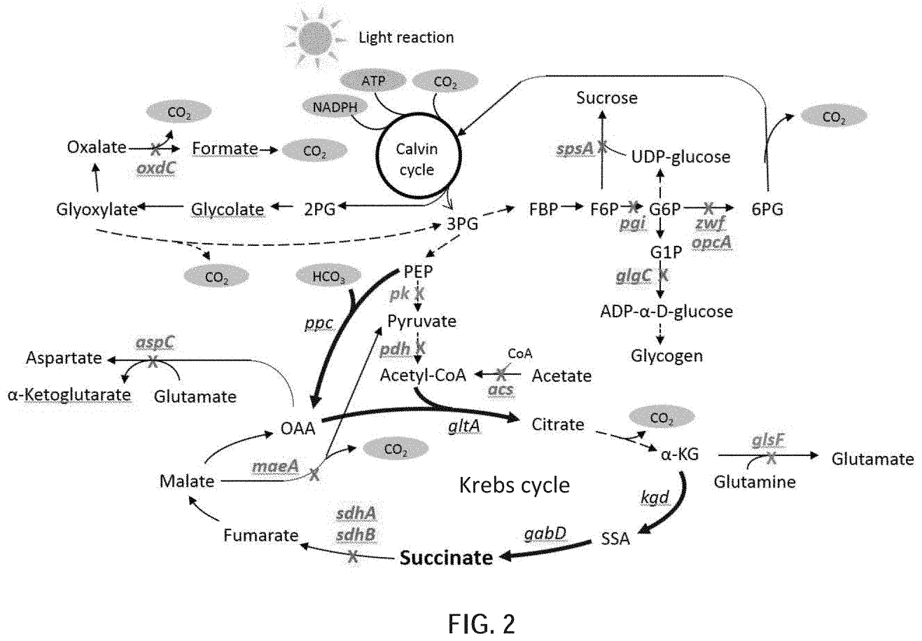

According to still another embodiment of the present invention, referring to FIG. 2, besides expressing α-ketoglutarate decarboxylase (Kgd), succinate semialdehyde dehydrogenase (GabD), citrate synthase (GltA), and phosphoenolpyruvate carboxylase (Ppc), various native genes are further suppressed in the host cell of the cyanobacterium strain. According to an embodiment of the present invention, with the suppression of at least one of the genes encoding glucose-1-phosphate adenylyltransferase (GlgC), succinate dehydrogenase subunit A (SdhA), and succinate dehydrogenase subunit B (SdhB), the production efficiency of succinate by the cyanobacterium strain can be further increased, while no adverse effect is caused to the growth of the host cell.

Particularly, FIG. 2 is a diagram showing the integration of the artificially constructed succinate production pathway as shown in FIG. 1 with other metabolic pathways in the host cell of cyanobacterium that may correlate with the artificially constructed pathway. In constructing transgenic strains according to various embodiments of the present invention, it has been experimentally found that the inhibition of different metabolic pathways (such as gene knockout or knockdown of the key enzyme in the metabolic pathway) greatly possibly causes the death or halted growth of the host cell. For example, it has been experimentally found that when one of the pyruvate kinase (pk) and ferredoxin-dependent glutamate synthase (GlsF) as shown in FIG. 2 is individually inhibited, the halted growth of the host cell is obviously caused, and thus the production of succinate is hindered since the host cell cannot be continuously cultured. In addition, although some correlating pathways compete with the succinate production pathway of the present invention, the succinate production cannot be enhanced or can only be enhanced to some limited extent when those correlating pathways is respectively suppressed. For example, it has been experimentally found that when any one of oxalate decarboxylase (oxdC), aspartate aminotransferase (aspC), malic enzyme A (maeA), pyruvate dehydrogenase (pdh), acetyl-CoA synthetase (acs), sucrose phosphate synthase A (spsA), phosphoglucose isomerase (pgi), glucose-6-phosphate dehydrogenase (zwf), and glucose-6-phosphate dehydrogenase assembly protein A (opcA) as shown in FIG. 2 is inhibited, the amount of succinate produced by the transgenic strain constructed with the succinate production pathway cannot be further increased. As stated above, in order to achieve the desired goal of increasing the succinate production while maintaining the normal function of the transgenic strain, the possible effects caused by any gene being knocked out, knocked down or inhibited need to be considered and confirmed.

According to some embodiments of the present invention, when at least one (or any one) of the genes encoding glucose-1-phosphate adenylyltransferase (GlgC), succinate dehydrogenase subunit A (SdhA), and succinate dehydrogenase subunit B (SdhB) is inhibited, the amount of succinate produced by the transgenic strain constructed with the succinate production pathway can be greatly increased while no significant adverse effects are caused to the host cells. According to a preferred embodiment of the present invention, when the expressions of the native genes encoding GlgC and SdhB in the host cell are both inhibited (double-inhibition strain), the succinate production is increased by about 1.7 times compared with the situation where no gene is inhibited (i.e., only a strain expressing Kgd, GabD, GltA, and Ppc, but having no inhibited native genes is constructed).

According to some embodiments of the present invention, various methods can be utilized to suppress the expression of the above genes. For example, at least one (or any one) of the genes encoding glucose-1-phosphate adenylyltransferase (GlgC), succinate dehydrogenase subunit A (SdhA), and succinate dehydrogenase subunit B (SdhB) can be inhibited by any conventional gene knockout technique or gene knockdown technique, or by adding an inhibitor. According to an embodiment of the present invention, knockdown of at least one of the genes encoding GlgC, SdhA, and SdhB in the transgenic strain constructed with the succinate production pathway is performed by CRISPRi for gene suppression. As described above, according to some embodiments, the genes encoding GlgC and SdhB can be knocked down by CRISPRi for gene suppression to increase the succinate production. However, the present invention can also achieve the above objectives by any other means

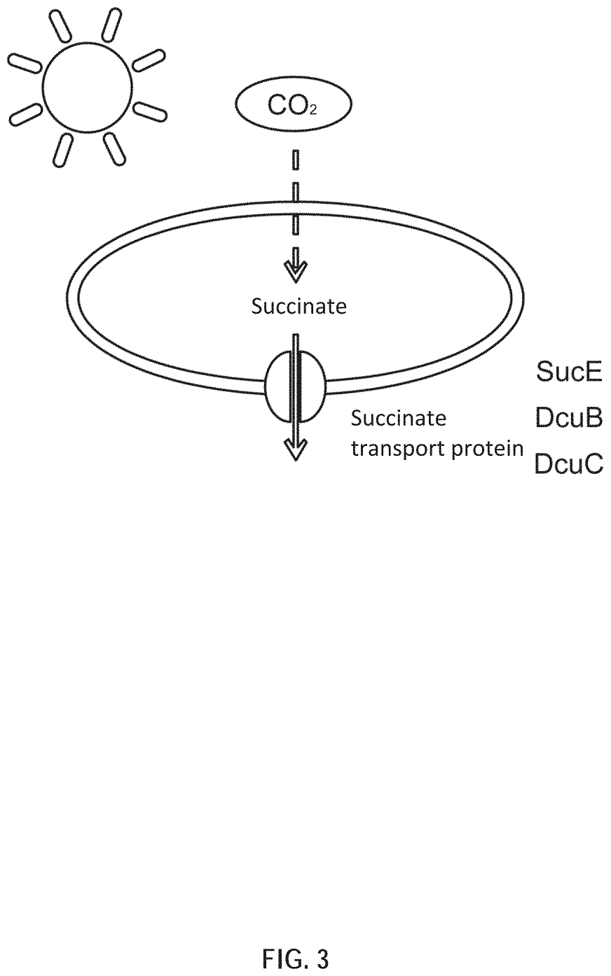

The succinate producing transgenic strain constructed according to various embodiments of the present invention is capable of consuming carbon dioxide to produce succinate under the condition of providing carbon dioxide and light, and excreting the produced succinate out of the host cell. Therefore, the desired succinate can be obtained directly by collecting the culture medium, with no need of collecting succinate concentrated in the cells by any procedure such as cell disruption/cell lysis or fermentation. However, according to some embodiments of the present invention, referring to FIG. 3, the gene encoding the succinate transport protein derived from other species may be further transfected into the host cell. In this way, the host cell can express the succinate transport protein, and the ratio and efficiency of succinate transport from the host cell to the external environment (such as the culture medium) are increased by the succinate transport protein, thereby further increasing the amount of succinate collected while the sustainable growth of the host cell is maintained. The transport protein is, for example, but not limited to, SucE from Corynebacterium glutamicum, and DcuB and DcuC from Escherichia coli.

According to some embodiments of the present invention, the autotrophic host cells constructed with the succinate production pathway according to various embodiments include Cyanobacteria such as Synechococcus elongatus PCC7942, Synechococcus elongatus UTEX 2973, Synechocystis sp. PCC6803, and Synechococcus sp. PCC7002. However, the present invention is not limited thereto. The host cell may be any cyanobacterium comprising native genes encoding GlgC, SdhA, and SdhB, and capable of producing phosphoenolpyruvate with carbon dioxide. Alternatively, the host cell may be any organisms comprising one of the native genes encoding GlgC, SdhA, and SdhB, and capable of converting carbon dioxide into phosphoenolpyruvate or undergoing photosynthesis. In view of the foregoing, according to the present invention, any organism having for example the same or similar metabolic pathway and mechanism, and thus capable of being constructing with and expressing a succinate production pathway in accordance with the same principles and concepts described herein, can be genetically modified to produce succinate based on the principle of the present invention.

All the transgenic strains described above with reference to various embodiments can undergo the Calvin cycle of photosynthesis and synthesize succinate through the artificially constructed succinate production pathway under the condition of providing light and carbon dioxide. Although the Calvin cycle can continue for a period of time without light, the operation of the Calvin cycle still requires the intermediates or enzymes produced or catalyzed by the light reactions in photosynthesis that can only proceed under light. Therefore, such transgenic strains can continue to produce succinate under light or for a short period of time after withdrawal of light, but it may be difficult to continue to produce succinate after a period of lack of light.

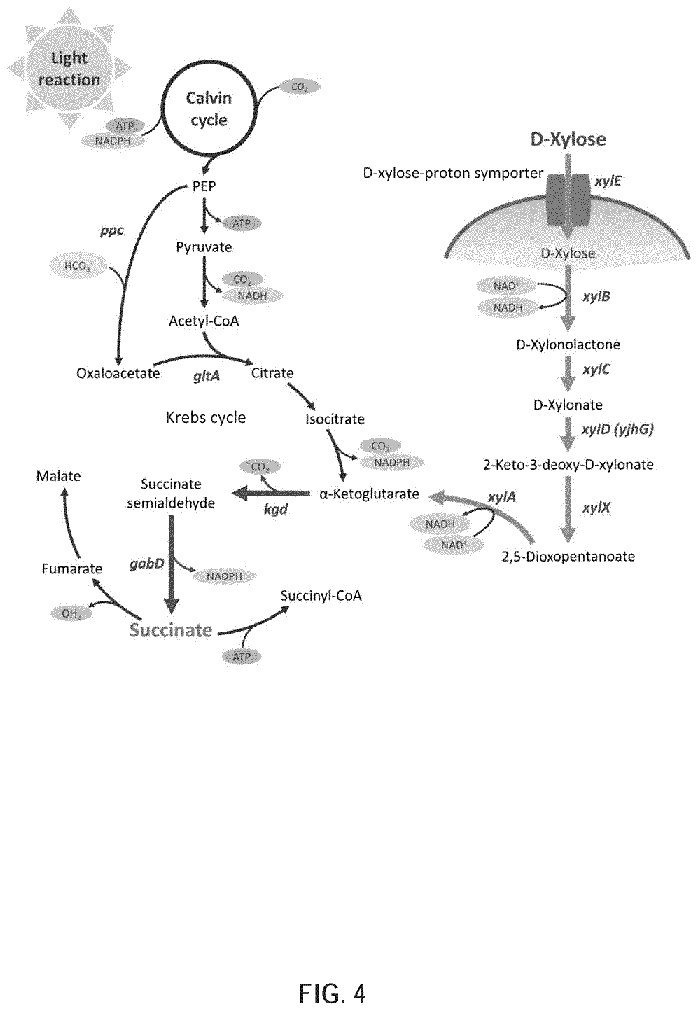

According to another embodiment of the present invention, the transgenic strain constructed with the succinate production pathway can be further genetically transformed to construct an additional xylose metabolic pathway. Specifically, referring to FIG. 4, in a transgenic strain that has the succinate production pathway and thus can converts α-ketoglutarate to succinate semialdehyde and then to succinate as described above, a pathway of converting xylose into α-ketoglutarate can be further constructed. In this way, succinate can be produced by providing xylose regardless of the light.

Particularly, in addition to the above-mentioned exogenous genes for constructing the succinate production pathway, other exogenous genes can be further transformed into the host cell to construct the xylose metabolic pathway. These exogenous genes include genes encoding D-xylose-proton symporter (xylE), D-xylose dehydrogenase (xylB), D-xylono-1,5-lactone lactonase (xylC), one of xylonate dehydratase (yjhG) or xylanase D (xylD), 2-keto-3-deoxyxylonate dehydratase (xylX), and α-ketoglutaric semialdehyde dehydrogenase (xylA). In the case of further transformation with the above-mentioned exogenous genes, referring to the right half of FIG. 4, when xylose is provided, the xylose can be transported through D-xylose-proton symporter on the cell membrane or cell wall of the host cell into the host cell, and then converted into α-ketoglutarate sequentially by D-xylose dehydrogenase (xylB), D-xylono-1,5-lactone lactonase (xylC), one of xylonate dehydratase (yjhG) or xylanase D (xylD), 2-keto-3-deoxyxylonate dehydratase (xylX), and α-ketoglutaric semialdehyde dehydrogenase (xylA). Therefore, the xylose metabolic pathway can be integrated with the succinate production pathway described above with reference to various embodiments, and a-ketoglutarate can be further converted into succinate by the α-ketoglutarate decarboxylase (Kgd) and succinate semialdehyde dehydrogenase (GabD). Therefore, in the presence of xylose, whether there is light or not, the transgenic strain is experimentally proved to be able to produce succinate by consuming xylose and excrete the produced succinate out of the host cell. Therefore, once xylose is provided, the transgenic strain having the succinate production pathway and the xylose metabolic pathway can continue to produce succinate under light and in the dark, thereby improving the efficiency and level of succinate production.

According to some embodiments of the present invention, the transgenic strain according to various embodiments above can be utilized to produce succinate. Specifically, the method for producing succinate comprises providing a transgenic strain according to various embodiments above, and culturing the transgenic strain under the condition of providing light and carbon dioxide. As a result, since the transgenic strains of the respective embodiments are constructed with a succinate production pathway, the transgenic strains according to various embodiments of the present invention can produce succinate by consuming carbon dioxide under the condition of providing light and carbon dioxide, regardless of whether or not the xylose metabolic pathway is additionally constructed. However, according to some other embodiments of the present invention, in the case that the transgenic strain is further structured to have the xylose metabolic pathway, it is also possible to produce succinate by alternately supplying carbon dioxide (in daytime and/or under light) and xylose (at night and/or in the dark) while the transgenic strain is cultured when the day and night alternate or the light and dark alternate. Therefore, succinate can be continuously produced at night and/or in the dark, thereby increasing the production of succinate and avoiding the interruption of succinate production. In addition, according to some embodiments of the present invention, xylose can also be provided to produce succinate when the day and night alternate or the light and dark alternate. Further, according to other embodiments of the present invention, if the providing of light and carbon dioxide is a first preset condition, and the providing of xylose is a second preset condition, the transgenic strain provided can be cultured under the first preset condition, the second preset condition and a combination thereof, so as to achieve the purpose of producing succinate.

According to some embodiments of the present invention, the first preset condition includes providing light at a light intensity of 100-800 uE, for example, 100 uE, 200 uE, 300 uE, 400 uE, 500 uE, 600 uE, 700 uE, and 800 uE. According to a preferred embodiment of the present invention, the first preset condition includes providing light at a light intensity of 200 uE. However, the above description is merely exemplary, the light may be provided at other light densities, and the present invention is not limited to the specific examples shown here.

According to some embodiments of the present invention, the providing of carbon dioxide in the first preset condition may include providing pure carbon dioxide, air containing carbon dioxide, or a chemical substance or liquid that can produce or be converted into carbon dioxide. Given that the transgenic strain cultured can receive carbon dioxide, any method may be used to provide carbon dioxide in accordance with the present invention, and the present invention is not limited to the specific examples shown here.

According to an embodiment of the present invention, the first preset condition can be achieved by introducing mixed air having a gas composition comprising 0 to 5% carbon dioxide at a flow rate of 20 to 80 cc/min to a culture medium in which the transgenic strain is cultured at a culture temperature of 30° C. However, this is only an example, and the culture temperature, gas composition, flow rate and other conditions can be adjusted according to the original characteristics of the autotrophic host cell, the characteristics of the genetically modified transgenic strain, and the cost. Various changes and modifications may be made by those skilled in the art from reading the foregoing and following description, and such changes and modifications should all fall within the scope of the present invention.

Since the transgenic strain according to various embodiments of the present invention can excrete succinate out of the cell after the production of succinate, the succinate can be obtained by collecting the culture medium or the like while the transgenic strain is continuously persistently cultured.

In summary, a transgenic strain that can produce succinate with a high throughput under continuous cultivation and a method for producing succinate are constructed, with which succinate critical to the food industry, industrial production and chemical industries can be produced by providing carbon dioxide. Further, the transgenic strain and the method for producing succinate according to the present invention can also consume excessively discharged carbon dioxide, thereby reducing the environmental problems caused by carbon dioxide at the time when succinate is produced.

### Definitions of Scientific and Technical Terms

As used herein, “succinate production pathway” is defined as a pathway by which carbon dioxide is converted into succinate through multiple metabolic processes and includes at least the metabolic processes involving the α-ketoglutarate decarboxylase (Kgd), succinate semialdehyde dehydrogenase (GabD), citrate synthase (GltA), and phosphoenolpyruvate carboxylase (Ppc), or proteins having α-ketoglutarate decarboxylase (Kgd), succinate semialdehyde dehydrogenase (GabD), citrate synthase (GltA), and phosphoenolpyruvate carboxylase (Ppc) activity.

As used herein, “xylose metabolic pathway” is defined as a pathway by which xylose is converted into a-ketoglutarate through multiple metabolic processes, and includes at least the metabolic processes involving D-xylose dehydrogenase (xylB), D-xylono-1,5-lactone lactonase (xylC), one of xylonate dehydratase (yjhG) or xylanase D (xylD), 2-keto-3-deoxyxylonate dehydratase (xylX), and α-ketoglutaric semialdehyde dehydrogenase (xylA), or proteins having the activities of these enzymes.

As used herein, “transgenic strain” is defined as a strain or cell line in which at least one exogenous gene (foreign gene) is transformed into the cell. The exogenous gene (foreign gene) may be present in the cell in any form. For example, it may be interposed in the genome or plasmid of a cell, interposed in an artificially constructed plasmid that is fed into the cell from the exterior, or interposed in any vector that is fed into the cell from the exterior.

As used herein, “host cell” refers to a cell of a transgenic strain that is used to be transformed with an exogenous gene (foreign gene).

As used herein, “exogenous gene (foreign gene)” is defined as a gene that does not originally exist in the host cell. The exogenous gene (foreign gene) may be a gene contained in an organism of the same species as or a different species from the host cell, or may be an artificially designed gene or a gene having a predetermined homology with the above gene. In some cases, the exogenous gene (foreign gene) may be identical to the original gene of the host cell itself, but is otherwise transferred into the host cell from the exterior.

### Specific Operation Examples

In accordance with the inventive principles of the present invention described above, some specific operation examples and results are listed below. However, it should be understood by those of ordinary skill in the art that the details of the operations described in the following specific operation examples are merely illustrative and exemplary of the embodiments of the present invention. Therefore, those skilled in the art can adjust or change the details of the operations and/or implement various embodiments that are not implemented in the following examples but are encompassed within the scope of the present invention with reference to the above description and the following examples. In addition, the details of the operations that are not specifically described in the following examples are details that can be easily adjusted or implemented according to the ordinary knowledge, and can be easily implemented by those of ordinary skilled in the art without undue experiments.

### Example 1: Chemicals and Reagents

All chemicals and reagents used in the examples of the present invention were purchased from Sigma-Aldrich (Saint Louis, Mo.), Amresco (Solon, Ohio) or J.T. Baker (Center Valley, Pa.) unless otherwise specified. Bacto-Agar was purchased from BD Bioscience. T4 DNA polymerase, KOD DNA polymerase and KOD Xtreme polymerase were purchased from New England Biolabs (Ipswich, Mass.) and END Millipore.

### Example 2: Culture Medium and Culture Conditions

Unless otherwise stated, all strains were cultured with modified BG-11 medium (a 1000× dilution of 1.5 g/L NaNO₃, 0.036 g/L CaCl₂.2H₂O, 0.012 g/L ammonium ferric citrate, 0.0022 g/L Na₂EDTA.2H₂O, 0.040 g/L K₂HPO₄, 0.070 g/L MgSO₄.7H₂O, 0.020 g/L Na₂CO₃, 0.00882 g/L sodium citrate, a trace mineral solution which comprises 1.43 g H3BO₃, 0.905 g MnCl₂.4H₂O, 0.111 g ZnSO₄.7H₂O, 0.195 g Na₂MoO₄.2H₂O, 0.039 g CuSO₄.5H₂O, and 0.0245 g Co(NO₃)₂.6H₂O in per 500 mL of water, and 50 mM NaHCO₃) under light.

Example 3: Culture of Cyanobacterium Strain

Since cyanobacterium is a relatively simple prokaryote among the autotrophic organisms, and the genetic modification method therefor is widely studied and developed. Therefore, cyanobacterium is mainly used as an experimental object in specific examples of the present invention. The cyanobacterium strains used in the present invention are shown in Table 1 below. All recombinant cyanobacterium strains were constructed and transformed with the plasmids listed in Table 2. The bacterium amount (cell amount) was monitored by measuring the optical density (OD) of the culture medium at a wavelength of 730 nm using the Biotek epoch 2 microplate spectrophotometer. The OD730 is calculated based on a recalibration of an optical path length 1 cm.

TABLE 1
Cyanobacterium strain

| Strain | Relevant genotype and descriptions |
|---|---|
| PCC7942 | Wild-type Synechococcus elongatus PCC 7942 |
| LAN1 | Ptrc::GabD, Kgd; SpecR inserted in NSI site of PCC 7942 |
| | chromosome |
| LAN2 | Ptrc::GabD, Kgd, GltA; SpecR inserted in NSI site of PCC |
| | 7942 chromosome |
| LAN3 | Ptrc::GabD, Kgd, GltA, ppc; SpecR inserted in NSI site of |
| | PCC 7942 chromosome |
| ML16 | PLacO1::dcas9, Ptrc::sgRNA(sdhA); kanR inserted in NSII |
| | site of LAN3 chromosome |
| ML19 | PLacO1::dcas9, Ptrc::sgRNA(sdhB); kanR inserted in NSII |
| | site of LAN3 chromosome |
| ML21 | PLacO1::dcas9, Ptrc::sgRNA(GlgC); kanR inserted in NSII |
| | site of LAN3 chromosome |
| CR8 | PLacO1::dcas9, Ptrc::sgRNA(GlgC, sdhB); kanR inserted in |
| | NSII site of LAN3 chromosome |
| RT51 | PLacO1::sucE; kanR inserted in NSII site of LAN3 |
| | chromosome |
| RT52 | PLacO1::dcuB; kanR inserted in NSII site of LAN3 |
| | chromosome |
| RT53 | PLacO1::dcuC; kanR inserted in NSII site of LAN3 |
| | chromosome |
| DN01 | PLacO1::xylA, xylX, xylB, xylC, yjhG, xylE; kanR inserted |
| | in NSII site of LAN3 chromosome |
| DN02 | PLacO1::xylA, xylX, xylB, xylC, xylD, xylE; kanR inserted |
| | in NSII site of LAN3 chromosome |
| UTEX2973 | Wide-type Synechococcus elongatus UTEX2973 |
| JTS01 | Ptrc::GabD, Kgd, GltA, ppc; SpecR inserted in NSI site of |
| | UTEX2973 chromosome |
| JTS02 | Wide-type Synechococcus elongatus UTEX2973 carrying |
| | plasmid pJTS02 |

TABLE 2
Plasmid

| Plasmid | Relevant genotype and descriptions |
|---|---|
| pSR3 | SpecR; NSI target (Lan et al., 2013), derived from pAM2991 |
| | Template for constructing pEL258, pEL259, and pEL260 |
| pEL258 | SpecR; NSI target; Ptrc::GabD, Kgd |
| pEL259 | SpecR; NSI target; Ptrc::GabD, Kgd, GltA |
| pEL260 | SpecR; NSI target; Ptrc::GabD, Kgd, GltA, Ppc |

### Example 4: Construction of Plasmid Comprising Exogenous Genes

The plasmid listed in Table 2 was stored and subcultured in E. coli XL-1 blue. The plasmid used in the present invention was constructed by Ligation-independent cloning (LIC). For the succinate production pathway, all the vectors containing NSI target were respectively amplified into two DNA fragments by PCR amplification. The vector fragment 1 was amplified by a specially designed primer using pSR3 as a template. The vector fragment 2 was amplified by a specially designed primer using pSR3 as a template. Then, the two fragments were assembled with other genes to form pEL258, pEL259 and pEL260. The gene used for assembly was one or more of GabD, Kgd, GltA, Ppc. Specifically, the gabD gene was derived from Escherichia coli, the Kgd gene was derived from Synechococcus elongatus PCC7002, and the GltA and Ppc gene were derived from Corynebacterium glutamicum. In addition, for the xylose metabolic pathway, all the vectors containing NSII target were respectively amplified into two DNA fragments by PCR amplification using specifically designed primers. Then the two DNA fragments were ligated into a plasmid and assembled with genes encoding D-xylose-proton symporter (xylE), D-xylose dehydrogenase (xylB), D-xylono-1,5-lactone lactonase (xylC), one of xylonate dehydratase (yjhG) or xylanase D (xylD), 2-keto-3-deoxyxylonate dehydratase (xylX), and α-ketoglutaric semialdehyde dehydrogenase (xylA) by Gibson assembly method. The genes encoding D-xylose-proton symporter (xylE), D-xylose dehydrogenase (xylB), D-xylono-1,5-lactone lactonase (xylC), one of xylonate dehydratase (yjhG) or xylanase D (xylD), 2-keto-3-deoxyxylonate dehydratase (xylX), and α-ketoglutaric semialdehyde dehydrogenase (xylA) can be defined as genes conventionally used to encode such enzymes or proteins, or genes that have 80% or higher homology to the genes encoding such enzymes or proteins and retaining the activity of such enzymes or proteins.

### Example 5: Construction of Plasmids by CRISPRi Technology

For the construction of a plasmid by the CRISPRi technology, a plasmid containing the gene encoding the dCAS9 protein and the sgRNA gene which guides the dCAS9 protein needed to be constructed. A suppression effect could be achieved by designing different sgRNA fragments to guide the dCas9 protein to the gene of a target intended to be suppressed. The sgRNA is, for example, the sequence as shown in SEQ ID NO: 5, and the 20 base pairs NNNNNNNNNNNNNNNNNNNN included therein represent the corresponding gene fragment of suppression, which varies depending on the gene target intended to be suppressed. Fragments of corresponding 20 base pairs used in this example for knockdown each one of the different genes are shown in Table 3 below. In Table 3, the designations 1 and 2 of the same gene represent different positions (inhibitory sites) for inhibition of this same gene. However, the foregoing description is merely exemplary, and the present invention is not limited thereto.

TABLE 3

| Inhibition of | SEQ ID | Inhibitory site |
|---|---|---|
| target gene | NO | (corresponding gene fragment) |
| sdhA-1 | 6 | GCTGCCAAGCCCCTGCCAGA |
| sdhA-2 | 7 | CGCCCTGAGCCGCCACGCTA |
| sdhB-1 | 8 | AGATCGTGACTGCAGGAATA |
| sdhB-2 | 9 | GGAGGCTTGCTGCAACGCAT |
| glgC-1 | 10 | TTGGCGCGCTGTTTGGTTAG |
| glgC-2 | 11 | TCAAGCGGTATTTGCCCGCC |
| maeA-1 | 12 | GAAACGCTGTGACTGGCATT |
| maeA-2 | 13 | GCTGATCGTCGTGAAACACC |
| aspC-1 | 14 | GCGATCGCGAGAGTCAACGA |
| aspC-2 | 15 | TCGGGATAGCTCAACCAGTA |
| glsF-1 | 16 | CAGCGTGGGCCTTGGTAGGT |
| glsF-2 | 17 | TCCCCAAGCCAACGCTCGAC |
| zwf-1 | 18 | GTTCAGGGACTTTGTCTTGG |
| zwf-2 | 19 | CTGGCGGCAAACGCCGTTCG |
| pgi-1 | 20 | GATCGTAGTAGAGCCAATCG |
| pgi-2 | 21 | CATCCGCTGTTCATCGGGGT |
| spsA-1 | 22 | CTGCCCTCGCAGCAGACCAT |
| spsA-2 | 23 | GACTTGTGGGGATTTAGCTT |
| pk-1 | 24 | GGACGTTGCAGGGCCAATCG |
| pk-2 | 25 | ATGATCGTCGTGGGTTCCGT |
| acsA-1 | 26 | GAAAAACCCGCTTCTCTTGG |
| acsA-2 | 27 | CAGTCCAGCGTCTGTTGCCA |
| pdhA-1 | 28 | GCCTGAGAAGCCTGAAAACT |
| pdhA-2 | 29 | CATGGCCTTGATAATGCCGC |
| pdhB-1 | 30 | TTCATCGATTGCAGCCCGTA |
| pdhB-2 | 31 | ACCGAAAGTCGCCGTACTTT |
| oxdC-1 | 32 | ACTACCTATCAGGACAAAAC |
| oxdC-2 | 33 | CCACCGTCGTAGTCCACAAG |
| opcA-1 | 34 | TCAGCCAGCGCGATTGGCGT |
| opcA-2 | 35 | TTCATAGACCACGATGCTGA |

### Example 6: Transformation of Cyanobacterium Strain and Gene Knockdown

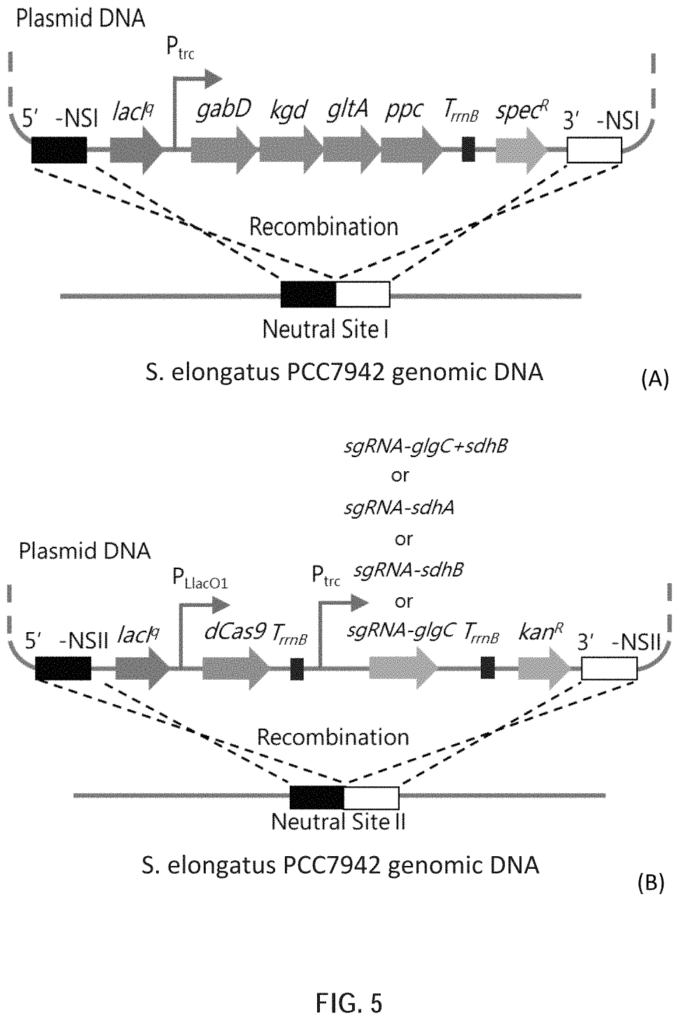

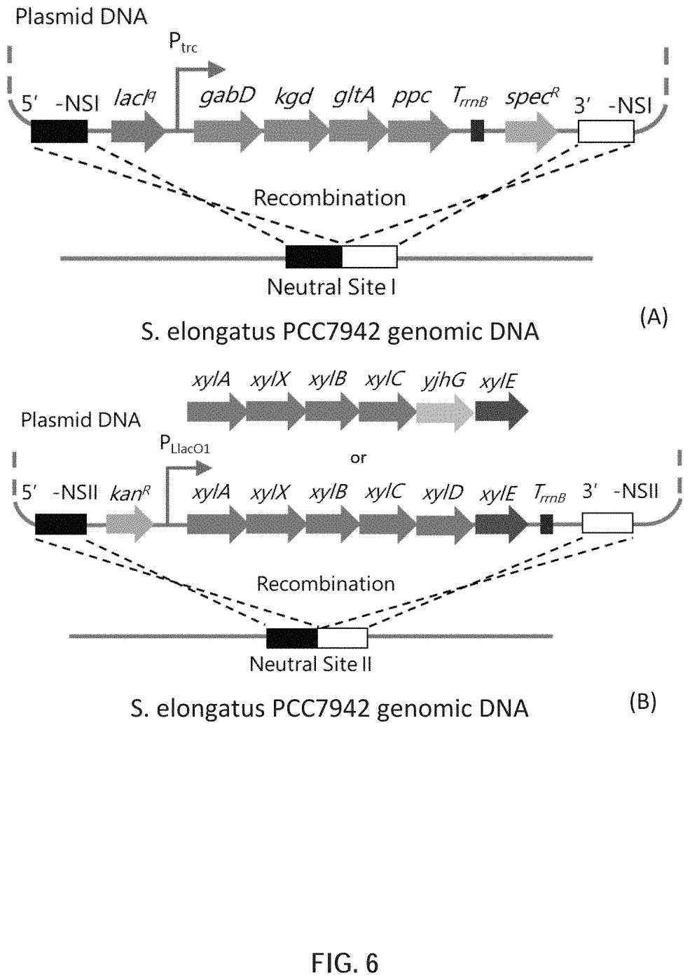

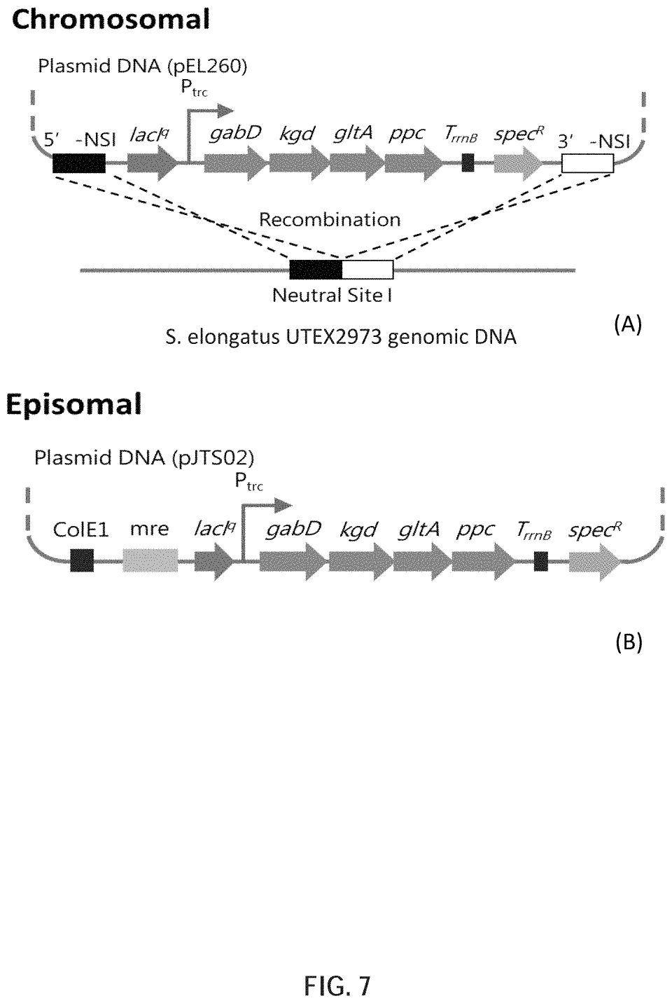

Generally, 50 ml of cyanobacterium was cultured to a concentration having an OD730 of 0.4 to 0.6, and then concentrated by centrifugation at 4000 xg to 2 ml. Next, 300 μL of the cyanobacterium at this concentration was co-incubated with 2 μg of the target plasmid DNA overnight in the dark. Then, a mixture of the cyanobacterium and the plasmid DNA was transferred to the BG-11 medium provided with Spectinomycin (20 μg/mL) for screening of the successfully transformed cyanobacterium. Therefore, the plasmid constructed in the above example was transformed into the host cell. The target exogenous gene could be interposed in the host cell by homologous recombination when the chromosome in the host cell has a specific recombination site. For example, FIGS. 5 and 6 show the construction of the succinate production pathway, the genes related to CRISPRi regulation and/or the xylose metabolic pathway in PCC7942. FIG. 5 shows the construction of the succinate production pathway and the genes related to CRISPRi regulation at different sites in the same PCC7942 strain. FIG. 6 shows the construction of the succinate production pathway and the xylose metabolic pathway at different sites in the same PCC7942 strain. However, the description here are merely experimental examples, and those of ordinary skill in the art can perform various transformations and gene knockdown of the cyanobacterium strain based on the principles of the present invention. For example, the construction of the succinate production pathways, the genes related to CRISPRi regulation, and the xylose metabolic pathways could be performed at different sites in the same specific cyanobacterium strain. In addition, in order to compare the aspects of introduction into the chromosome and simple transformation into the host cell, according to an embodiment of the present invention, the succinate production pathway was constructed in different ways by respectively transforming UTEX2973 with a plasmid introduced into the chromosome or cytoplasm of the UTEX2973 strain, for example, as shown in FIG. 7.

### Example 7: DNA Purification, Amplification and Sequencing

The genomic DNA of cyanobacterium strain was purified using the Wizard® genomic DNA purification kit purchased from Promega, according to the manufacturer's operation manual. The genomic DNA was used for the chromosomal segregation check. The complete chromosome isolation of the genetically engineered strain was confirmed by PCR using primers that bind to the NSI homologous site or the NSII homologous site, where the primers could be designed by a conventional technique based on the sequence of the NSI homologous site or the NSII homologous site. Thus, the details would not be described here. As a result, the strains that have been confirmed to have the expected gene sequence (successful transformation and/or gene knockout or knockdown) proceeded the following analysis and test. Moreover, the detection methods for the transformation of exogenous genes into the cells or introduction of the exogenous genes in the chromosomes of the host cell may be performed with any conventional or commercial kit or method, or any method developed in the future. The specific examples described herein are merely exemplary, and the present invention can be practiced by various applicable methods.

### Example 8: Test of the Toxicity of Succinate to Cyanobacterium and the Succinate Consumption when Culturing Cyanobacterium

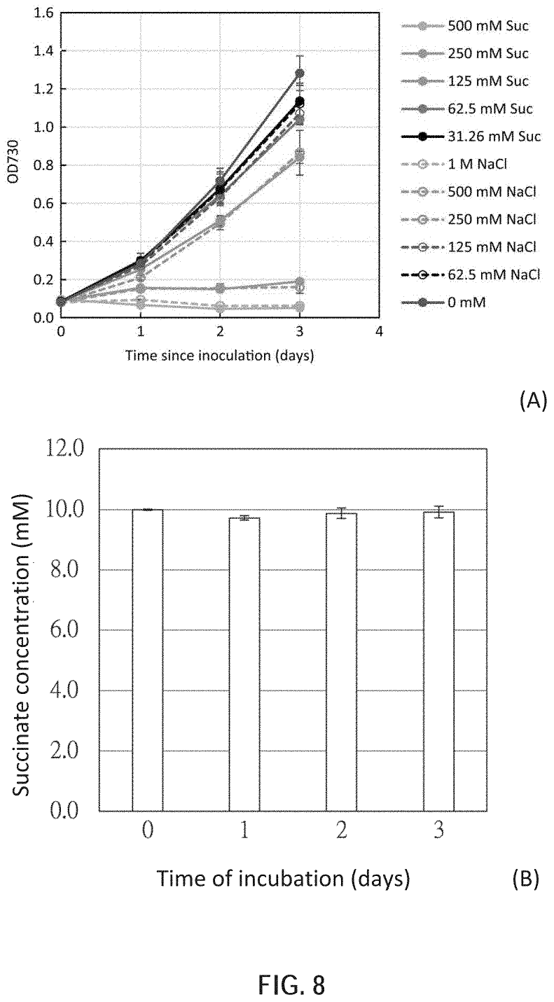

To assess whether succinate is toxic to the PCC7942 cyanobacterium strain, wild-type PCC7942 was cultured in BG-11 medium, and different concentrations of disodium succinate for the experimental group or sodium chloride for the control group were added. Next, the cell density of PCC7942 was observed for three days. The results are shown in panel A of FIG. 8. The growth inhibition effect of disodium succinate on the cyanobacterium strain is almost equivalent to the growth inhibition effect of sodium chloride on the cyanobacterium strain containing the same concentration of sodium. The growth inhibition effect on the cyanobacterium strain shown in Panel A of FIG. 8 can be attributed to the sodium intolerance. Therefore, it can be seen from the results that succinate itself is not toxic to the growth or persistence of the cyanobacterium strain. In addition, in order to investigate whether succinate will be consumed by the cyanobacterium strain, wild-type PCC7942 with a cell density of OD730 of about 1 was cultured in BG-11 medium, in which 10 mM succinate was added. Then, the change in succinate concentration was observed for three days. The result is shown in Panel B of FIG. 8. It can be seen from the results that the PCC7942 cyanobacterium strain can grow without consuming succinate.

### Example 9: Succinate Production Test of Transgenic Strain Involving the Exogenous Genes of KGd, GabD, GltA, and Ppc in Succinate Production Pathway

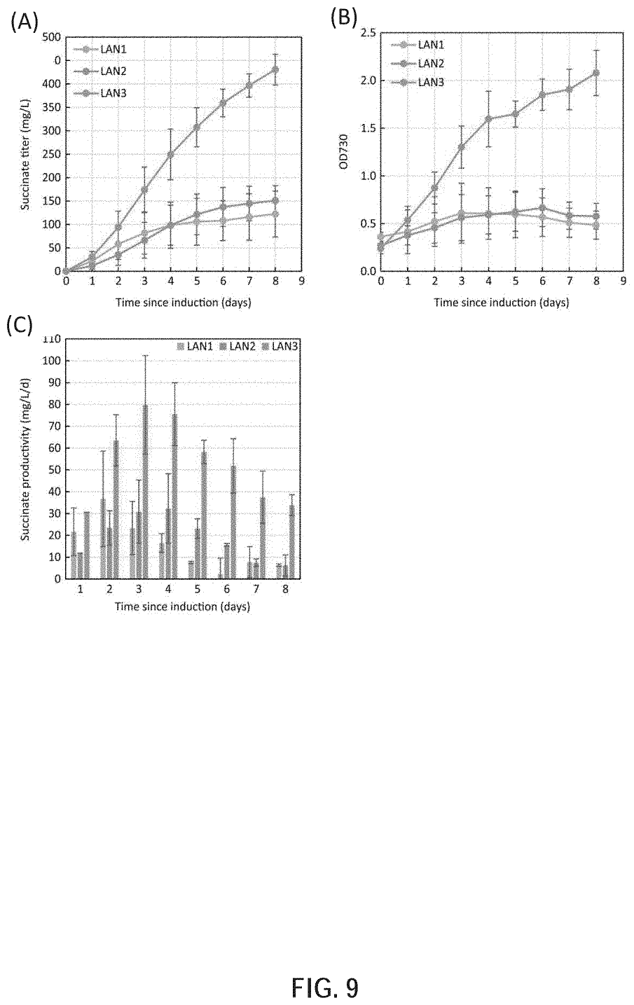

As shown in Table 1, PCC7942 cell lines LAN1, LAN2, and LAN3 transformed to comprise two or more of the exogenous genes of Kgd, GabD, GltA, and Ppc were respectively cultured in 40 mL BG-11, in which 50 mM NaHCO₃ (decomposable into carbon dioxide) and Spectinomycin (20 μg/mL) were provided. The initial cell density in OD730 was about 0.03-0.05. After three days of culture, OD730 was about 0.3-0.5, and the promoter expression was induced by 0.1 nM IPTG. Next, a portion of the culture sample (1 mL, corresponding to 2.5% of the volume of the culture) was taken from the culture medium daily for testing the succinate concentration and cell density. After the sample was taken, 1 mL of BG-11 provided with 500 mM NaHCO₃, Spectinomycin (20 μg/mL), and IPTG (0.1 mM) were re-added to the medium. The measured cell density and succinate concentration produced are shown in FIG. 9. The LAN3 strain transformed with all of Kgd, GabD, GltA, and Ppc has greatly enhanced succinate concentration produced and cell density of the strain, compared with the LAN1 and LAN2 strains transformed only with some of Kgd, GabD, GltA, and Ppc. As can be seen from the results in FIG. 9, compared to other transgenic strains, the LAN3 strain transformed with all of Kgd, GabD, GltA, and Ppc has sustained a higher growth by further reducing or avoiding the influence, changing and even breaking the physiological balance of the host cell itself, and greatly enhance the potency and throughput of succinate production at the same time.

### Example 10: Test of Succinate Production by Transgenic Strains Constructed with Succinate Production Pathway in which Genes in a Competitive Pathway are Further Inhibited

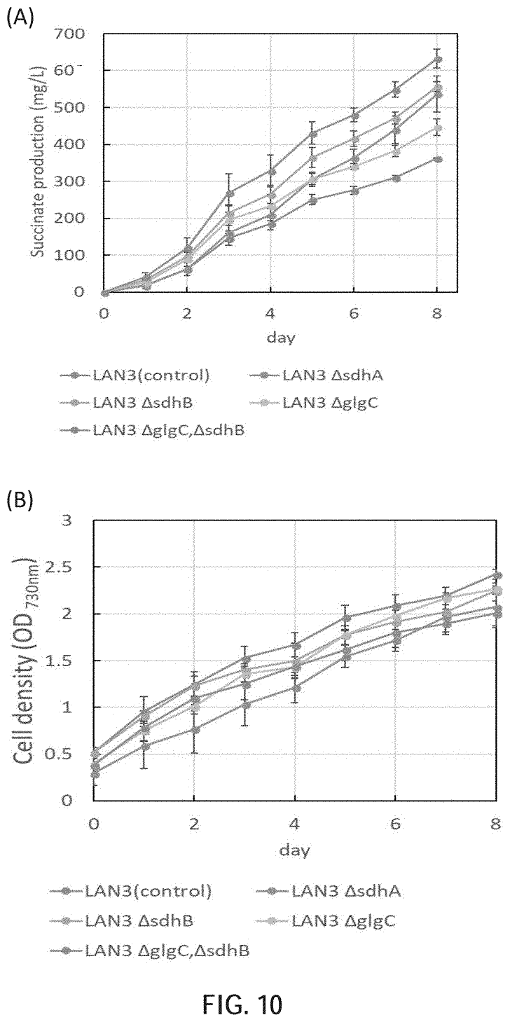

As shown in Table 1, in the PCC7942 strain LAN3, at least one of the native genes encoding pk, oxdC, aspC, maeA, pdh, acs, spsA, pgi, zwf, opcA, GlsF, GlgC, SdhA, and SdhB in the host cell was further inhibited (knocked down), and various strains produced were respectively cultured in 40 mL BG-11 provided with 50 mM NaHCO₃, Spectinomycin (20 μg/mL) and Kanamycin (10 μg/mL). The initial cell density in OD730 was about 0.03-0.05. After three days of culture, OD730 was about 0.3-0.5, and the promoter expression was induced by 0.1 nM IPTG. Next, a portion of the culture sample (1 mL, corresponding to 2.5% of the volume of the culture) was taken from the culture medium daily for testing the succinate concentration and cell density. After the sample was taken, 1 mL of BG-11 provided with 500 mM NaHCO₃, Spectinomycin (20 μg/mL), Kanamycin (10 μg/mL) and IPTG (0.1 mM) were added to the medium. According to the result of culture in one embodiment of the present invention, if one of pk and oxdC is inhibited, the growth of host cell is greatly retarded (not shown). In addition, if one of oxdC, aspC, maeA, pdh, acs, spsA, pgi, zwf, opcA, and GlsF is inhibited, the succinate production can not or hardly improved (not shown). Finally, according to some embodiments of the present invention, when at least one of the genes of GlgC, SdhA, and SdhB is suppressed, the amount of succinate produced can be significantly improved compared to LAN3. In particular, when both GlgC and SdhB are suppressed, the succinate produced can be maximized. On day 8 of culture, the strains with inhibited GlgC and SdhB can produce succinate in an amount of 632 mg/L, which is 1.7 times of 362 mg/L of the uninhibited strain. The above results from inhibition of at least one of the genes of GlgC, SdhA, and SdhB are shown in FIG. 10.

### Example 11: Test of Succinate Production by Transgenic Strains Constructed with Succinate Production Pathway in which a Succinate Transport Protein is Further Expressed

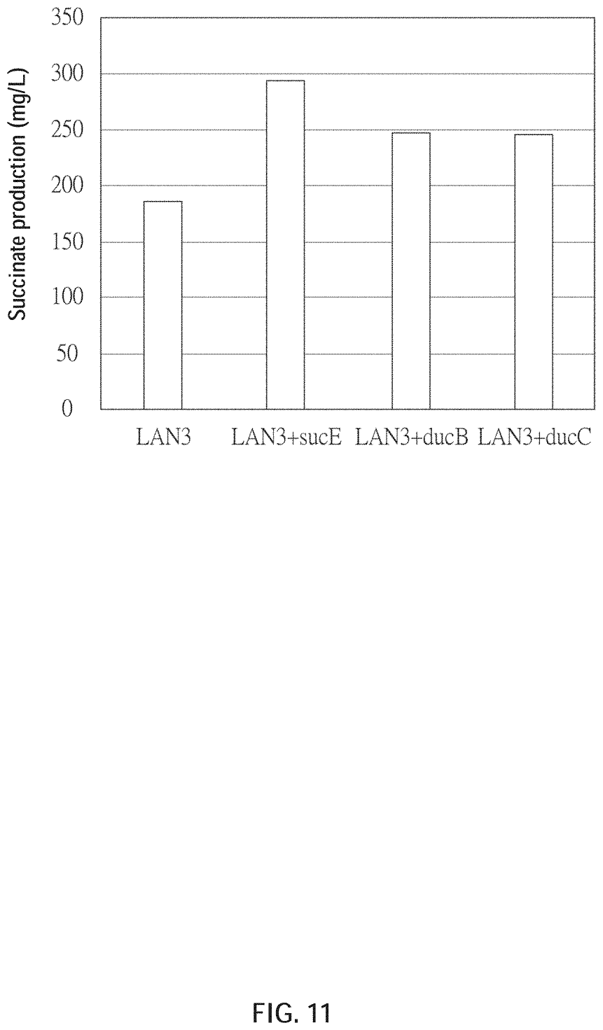

As shown in Table 1, the PCC7942 strain LAN3 was further transformed with an exogenous gene of the succinate transport protein sucE, dcuB or dcuC (for the method of transferring and transforming the gene of the succinate transport protein into the strain, see the way of transferring and transforming other exogenous genes stated above or other conventional methods). SucE was derived from Corynebacterium glutamicum, and DcuB and DcuC were derived from Escherichia coli. Various strains produced were respectively cultured in 40 mL BG-11 provided with 50 mM NaHCO₃, Spectinomycin (20 μg/mL) and Kanamycin (10 μg/mL). The initial cell density in OD730 was about 0.03-0.05. After three days of culture, OD730 was about 0.3-0.5, and the promoter expression was induced by 0.1 nM IPTG. Next, a portion of the culture sample (1 mL, corresponding to 2.5% of the volume of the culture) was taken from the culture medium daily for testing the succinate concentration and cell density. After the sample was taken, 1 mL of BG-11 provided with 500 mM NaHCO₃, Spectinomycin (20 μg/mL), Kanamycin (10 μg/mL) and IPTG (0.1 mM) were re-added to the medium. The results of the succinate production by each of the cultured strains are shown in FIG. 11, in which the strains expressing the succinate transport protein respectively produce 293 mg/L (LAN3+sucE), 247 mg/L (LAN3+dcuB), 245 mg/L (LAN3+dcuC) in six days, which is 1.58, 1.33, 1.32 times of that of the strain LAN3 (185 mg/L) which does not express the transport protein.

### Example 12: Test of Succinate Production at Night by Transgenic Strains Constructed with Succinate Production Pathway

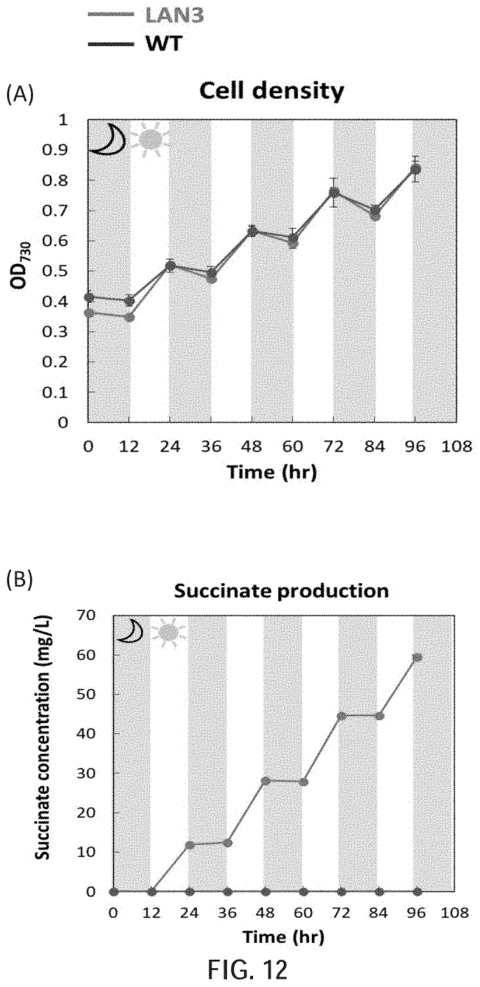

LAN3 and wide-type PCC7942 were respectively cultured in 40 mL BG-11 provided with 50 mM NaHCO₃, Spectinomycin (20 μg/mL) and Kanamycin (10 μg/mL). The initial cell density in OD730 was about 0.03-0.05. The promoter expression was induced by 0.1 nM IPTG at first. Next, the strain was alternately culture under light (200 μE) and in the dark (light and dark for 12 hrs each), and a portion of the culture sample (1 mL, corresponding to 2.5% of the volume of the culture) was taken from the culture medium every 1 hr for testing the succinate concentration and cell density. After the sample was taken, 1 mL of BG-11 provided with 500 mM NaHCO₃, Spectinomycin (20 μg/mL), Kanamycin (10 μg/mL) and IPTG (0.1 mM) were re-added to the medium. The succinate concentration and cell density from 0 to 96 hours are shown in FIG. 12. It can be seen from the results that the succinate production strain constructed with the succinate production pathway cannot produce succinate at night without using any additional organic carbon sources.

### Example 13: Test of Succinate Production at Night by Transgenic Strains Constructed with Succinate Production Pathway and Xylose Metabolic Pathway

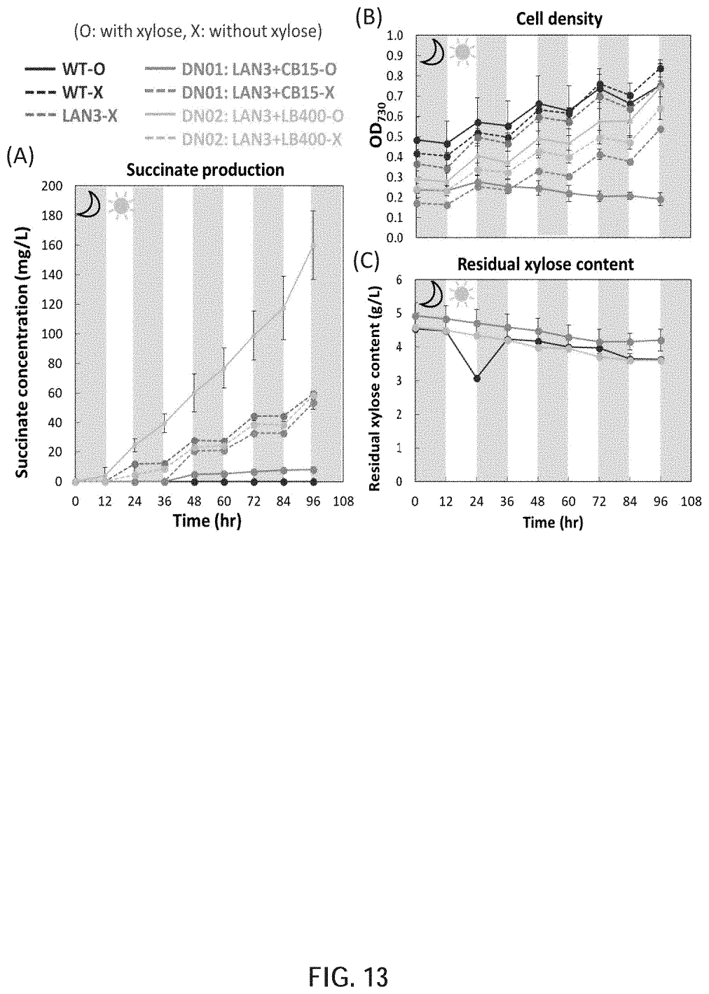

As shown in Table 1, wide-type PCC7942, LAN3, DN01 and DN02 were respectively cultured in 40 mL BG-11 provided with 50 mM NaHCO₃, Spectinomycin (20 μg/mL), Kanamycin (10 μg/mL), and 5 g/L xylose. The initial cell density in OD730 was about 0.03-0.05. The promoter expression was induced by 0.1 nM IPTG at first. Next, the strain was alternately culture under light (200 μE) and in the dark (light and dark for 12 hrs each), and a portion of the culture sample (1 mL, corresponding to 2.5% of the volume of the culture) was taken from the culture medium every 1 hr for testing the succinate concentration and cell density. After the sample was taken, 1 mL of BG-11 provided with 500 mM NaHCO₃, Spectinomycin (20 μg/mL), Kanamycin (10 μg/mL) and IPTG (0.1 mM) were re-added to the medium. The succinate production, cell density, and residual xylose content observed under light and at night during 0-96 hrs are shown in FIG. 13. The cyanobacterium DN02, which expresses the xylose metabolic pathway, can produce succinate with xylose at night, and achieves a yield of succinate of up to 160 mg/L in four days. From the above results, it can be seen that the cyanobacterium strain constructed with the succinate production pathway and further expressing the xylose metabolic pathway can produce succinate with xylose in the absence of light, thereby greatly increasing the succinate production by the transgenic strain.

### Example 14: Test of Succinate Production by Transgenic Strains with Succinate Production Pathway in which Exogenous Genes are Interposed in the Genome or in the Cytoplasmic Plasmid (Episomal)

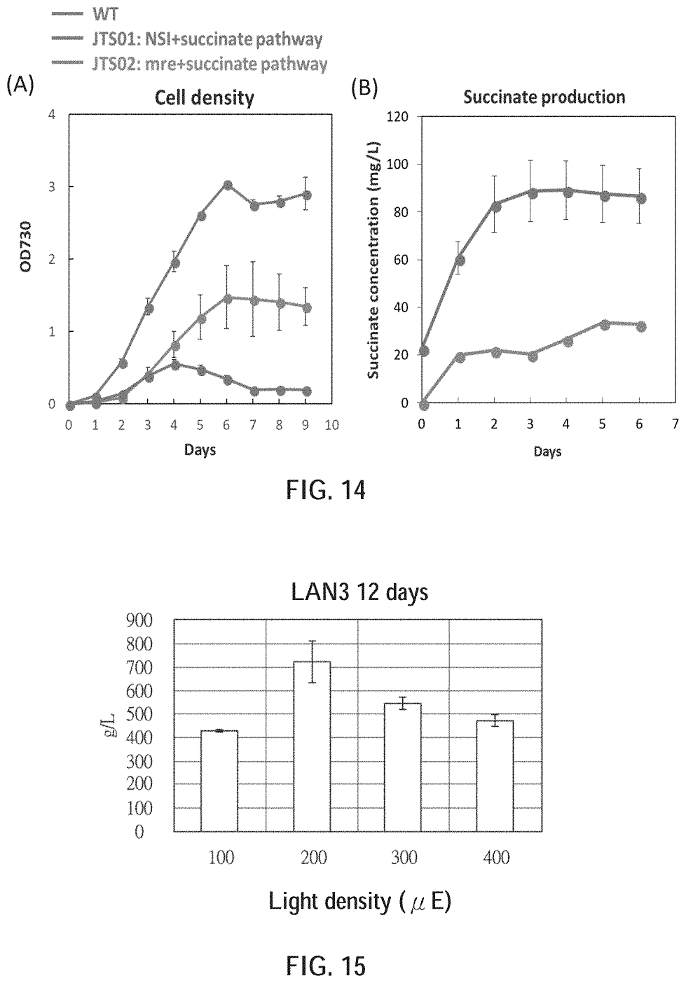

As shown in Table 1, JTS01, JTS02 and wide-type PCC7942 were respectively cultured in 40 mL BG-11 provided with 50 mM NaHCO₃ and Spectinomycin (20 μg/mL). The initial cell density in OD730 was about 0.03-0.05. After three days of culture, OD730 was about 0.3-0.5, and the promoter expression was induced by 0.1 nM IPTG. Next, a portion of the culture sample (1 mL, corresponding to 2.5% of the volume of the culture) was taken from the culture medium daily for testing the succinate concentration and cell density. After the sample was taken, 1 mL of BG-11 provided with 500 mM NaHCO₃, Spectinomycin (20 μg/mL), and IPTG (0.1 mM) were re-added to the medium. The cell density and succinate production of each strain cultivated are shown in FIG. 14. Whether the exogenous genes are transformed by interposing into the genome or as a cytoplasmic plasmid (Episomal), the strains JTS01 and JTS02 constructed with the succinate production pathway both can successfully produce succinate in an amount of about 80 mg/L and 20 mg/L (day 6), respectively. It is thus proved that as long as the enzyme or protein required for the succinate production pathway of the present invention can be expressed, the way of transforming the exogenous genes into the host cell can be varied.

### Example 15: Test of Succinate Production by Transgenic Strains Constructed with Succinate Production Pathway Cultured at Various Light Intensities by Directly Introducing Gas Containing Carbon Dioxide

Based on Example 8, excess sodium would have an inhibitory effect on the growth of transgenic strain and the succinate production associated with the cell amount of the transgenic strain. Therefore, according to an embodiment of the present invention, sodium bicarbonate was further replaced by introducing a gas containing carbon dioxide into the culture medium (in some examples, carbon dioxide can be provided by providing sodium bicarbonate which is decomposed to form carbon dioxide), thereby reducing the inhibition effect resulted from the presence of sodium on the cell amount and succinate production. As shown in Table 1, LAN3 was inoculated in 60 mL BG-11, and cultured at various light intensities (100, 200, 300, and 400 μE) by introducing mixed air having a gas composition comprising 0 to 5% carbon dioxide at a flow rate of 20 to 80 cc/min to a culture medium provided with Spectinomycin (20 μg/mL) in which the transgenic strain is cultured at a culture temperature of 30° C. The initial cell density in OD730 was about 0.03-0.05. After three days of culture, OD730 was about 0.3-0.5, and the promoter expression was induced by 0.1 nM IPTG. Next, a culture sample (1 mL, corresponding to 2.5% of the volume of the culture) was taken from the culture medium after 12 days for testing the succinate concentration and cell density. The succinate production of each strain cultivated at various light intensities is shown in FIG. 15. The succinate production strain LAN3 is tested to have the highest production of 700 mg/L on day 12 at a light intensity of 200 μE. It should be noted that this is only a preferred light intensity according to an embodiment of the present invention, and the light intensity used to promote the production of succinate is not limited thereto. Based on this example, a light intensity of 100, 200, 300, 400 μE or a value in the range therebetween can be used to promote the succinate production. However, the present invention is not limited thereto. For example, a light intensity greater than 400 μE, such as 500, 600, 700, and 800 μE, or a value in the range therebetween, can also be used to promote the succinate production.

### Example 16: Test of Succinate Production by Transgenic Strains Constructed with Succinate Production Pathway Cultured at a Light Intensity of 200 μE by Directly Introducing Gas Containing Carbon Dioxide

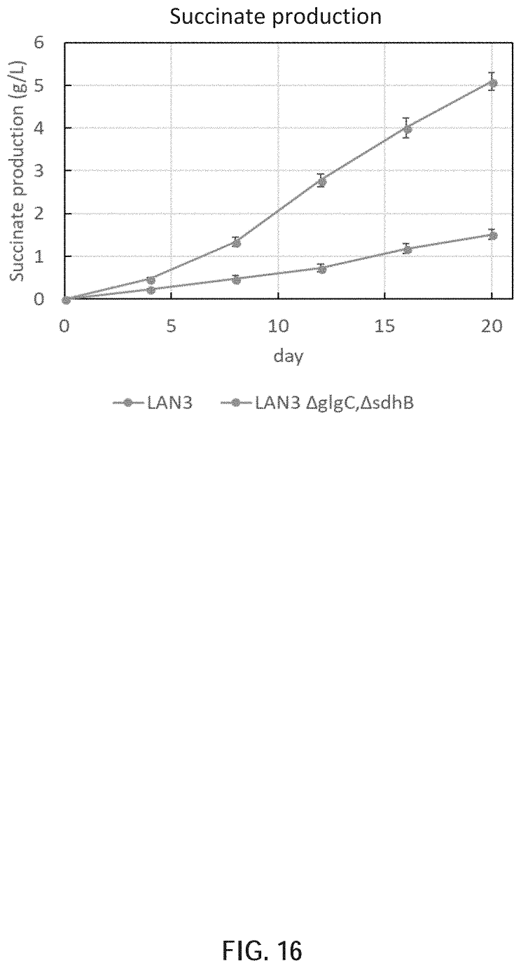

As shown in Table 1, LAN3 and CR8 were inoculated in 60 mL BG-11, and cultured at a light intensity of 200 μE by introducing mixed air having a gas composition comprising 0 to 5% carbon dioxide at a flow rate of 20 to 80 cc/min to a culture medium provided with Spectinomycin (20 μg/mL) and Kanamycin (10 μg/mL) in which the transgenic strain is cultured at a culture temperature of 30° C. The initial cell density in OD730 was about 0.03-0.05. After three days of culture, OD730 was about 0.3-0.5, and the promoter expression was induced by 0.1 nM IPTG. Next, a portion of the culture sample (1 mL, corresponding to 2.5% of the volume of the culture) was taken from the culture medium daily for testing the succinate concentration and cell density. After the sample was taken, 1 mL of BG-11 provided with Spectinomycin (20 μg/mL), Kanamycin (10 μg/mL) and IPTG (0.1 mM) were re-added to the medium. The succinate production of each strain cultivated is shown in FIG. 16. It can be seen from FIG. 16 that the strain CR8 constructed with the succinate production pathway and having inhibited GlgC and SdhB of the host cell has a high succinate production of about 5 g/L on day 20, which is further increased by about 3 times, compared with the strain LAN3 in which GlgC and SdhB are not inhibited.

### Example 17: Determination of Succinate

In the above example, the collected sample (1 mL) was centrifuged at 20,000 xg for 10 min. The supernatant was analyzed by Agilent 1260 Infinity HPLC equipped with an Agilent Hi-Plex-H (700×7.7 mm) organic acid analytical column. The Bio-Rad Micro-Guard Cation H protection column (30×4.6 mm) was connected in tandem before the analytical column. Succinate was detected by detecting the absorbance at 210 nm by a photodiode array detector. The column was maintained at 65° C. throughout the HPLC process. The injection volume used was 20 μL. The mobile phase used was 5 mM H₂SO₄ at a constant flow rate of 0.6 mL/min. The concentration of succinate in the culture medium was quantified based on a standard curve of the results of HPLC analysis of 0.1-10 mM standard succinate solutions. The fumarate, a by-product in the production of succinate, was quantified based on a standard curve established from standard solutions with a concentration of 10 μM to 1 mM.

In summary, each of the transgenic strains constructed according to the present invention and the method for producing succinate with such transgenic strains can produce succinate by providing carbon dioxide or a precursor or raw material able to produce carbon dioxide (that is, providing carbon dioxide by producing carbon dioxide) under light. Therefore, this reduces the dependence on petrochemical raw materials in the production of succinate, the cost of raw materials, and also the negative impact of carbon dioxide on the environment. Further, the produced succinate can be collected by collecting the culture medium outside the cells. Therefore, it is possible to produce and collect succinate in a single step while the transgenic strain is still cultured (without additional cell disruption/cell lysis, terminating the growth of the cells, killing the cells, or transporting and collecting the products inside the cell), thereby improving the production efficiency and simplifying the production process. As described above, the transgenic strain and the method for producing succinate according to the present invention have the potential of industrial mass production and wide use in various industries.

The above description is only some preferred embodiments of the present invention. It should be noted that various changes and modifications can be made to the present invention without departing from the spirit and scope of the invention. It will be apparent to those skilled in the art that the present invention is defined by the scope of the appended claims, and that various changes, combinations, modifications, and alterations are possible without departing from spirit and scope defined by the appended claims of the present invention.

### REFERENCES

1. Ethan I. Lan, Crystal T. Wei, 2016. Metabolic engineering of cyanobacteria for the photosynthetic production of succinate. Metabolic Engineering, 38, 483-493

2. Lin, H., Bennett, G. N., San, K. Y., 2005a. Genetic reconstruction of the aerobic central metabolism in Escherichia coli for the absolute aerobic production of succinate. Biotechnol Bioeng 89, 148-156.

3. Lin, H., Bennett, G. N., San, K. Y., 2005b. Metabolic engineering of aerobic succinate production systems in Escherichia coli to improve process productivity and achieve the maximum theoretical succinate yield. Metabolic engineering 7, 116-127.

4. Beauprez, J. J., De Mey, M., Soetaert, W. K., 2010. Microbial succinic acid production: Natural versus metabolic engineered producers. Process Biochem 45, 1103-1114.

5. Blankschien, M. D., Clomburg, J., Gonzalez, R., 2010. Metabolic engineering of Escherichia coli for the production of succinate from glycerol. Metabolic engineering 12, 409-419.

6. Lee, P. C., Lee, S. Y., Hong, S. H., Chang, H. N., 2002. Isolation and characterization of a new succinic acid-producing bacterium, Mannheimia succiniciproducens MBEL55E, from bovine rumen. Applied microbiology and biotechnology 58, 663-668.

7. Li, N., Zhang, B., Chen, T., Wang, Z. W., Tang, Y. J., Zhao, X. M., 2013. Directed pathway evolution of the glyoxylate shunt in Escherichia coli for improved aerobic succinate production from glycerol. J Ind Microbiol Biot 40, 1461-1475.

8. Li, Y., Huang, B., Wu, H., Li, Z., Ye, Q., Zhang, Y. P., 2016. Production of succinate from acetate by metabolically engineered Escherichia coli. Acs Synth Biol.

9. Nghiem, N. P., Davison, B. H., Suttle, B. E., Richardson, G. R., 1997. Production of succinic acid by Anaerobiospirillum succiniciproducens. Applied biochemistry and biotechnology 63-5, 565-576.

10. Okino, S., Noburyu, R., Suda, M., Jojima, T., Inui, M., Yukawa, H., 2008. An efficient succinic acid production process in a metabolically engineered Corynebacterium glutamicum strain. Applied microbiology and biotechnology 81, 459-464.

11. Stols, L., Donnelly, M. I., 1997. Production of succinic acid through overexpression of NAD(+)-dependent malic enzyme in an Escherichia coli mutant. Applied and Environmental Microbiology 63, 2695-2701.

12. Wang, J., Zhu, J., Bennett, G. N., San, K. Y., 2011. Succinate production from different carbon sources under anaerobic conditions by metabolic engineered Escherichia coli strains. Metabolic engineering 13, 328-335.

13. Zhang, X. L., Jantama, K., Moore, J. C., Jarboe, L. R., Shanmugam, K. T., Ingram, L. O., 2009. Metabolic evolution of energy-conserving pathways for succinate production in Escherichia coli. Proceedings of the National Academy of Sciences of the United States of America 106, 20180-20185.

14. Zheng, Z., Chen, T., Zhao, M., Wang, Z., Zhao, X., 2012. Engineering Escherichia coli for succinate production from hemicellulose via consolidated bioprocessing. Microb Cell Fact 11, 37.

15. Zhu, L. W., Li, X. H., Zhang, L., Li, H. M., Liu, J. H., Yuan, Z. P., Chen, T., Tang, Y. J., 2013. Activation of glyoxylate pathway without the activation of its related gene in succinate-producing engineered Escherichia coli. Metabolic engineering 20, 9-19.

16. Zhang, S., Bryant, D. A., 2011. The tricarboxylic acid cycle in cyanobacteria. Science 334, 1551-1553.

17. Chun-Hung Huang, Claire R. Shen, Hung Li, Li-Yu Sung, Meng-Ying Wu and Yu-Chen Hu., 2016. CRISPR interference (CRISPRi) for gene regulation and succinate production in cyanobacterium S. elongatus PCC 7942. Microb Cell Fact 15, 196.

18. Hung Li, Claire R. Shen, Chun-Hung Huang, Li-Yu Sung, Meng-Ying Wu, Yu-Chen Hu., 2016. CRISPR-Cas9 for the genome engineering of cyanobacteria and succinate production. Metabolic engineering 38, 293-302

1. A transgenic strain for producing succinate, comprising:
an autotrophic host cell; and
a plurality of exogenous genes within the host cell, comprising:
a first exogenous gene, which is a gene encoding a-ketoglutarate decarboxylase (Kgd) or a gene having 80% or higher sequence identity to SEQ ID NO: 1 and having Kgd activity;
a second exogenous gene, which is a gene encoding succinate semialdehyde dehydrogenase (GabD) or a gene having 80% or higher sequence identity to SEQ ID NO: 2 and having GabD activity;
a third exogenous gene, which is a gene encoding citrate synthase (GltA) or a gene having 80% or higher sequence identity to SEQ ID NO: 3 and having GltA activity; and
a fourth exogenous gene, which is a gene encoding phosphoenolpyruvate carboxylase (Ppc) or a gene having 80% or higher sequence identity to SEQ ID NO: 4 and having Ppc activity,
wherein expression of at least one of native genes encoding glucose-1-phosphate adenylyltransferase (GlgC), succinate dehydrogenase subunit A (SdhA), and succinate dehydrogenase subunit B (SdhB) is suppressed in the host cell.

2. The transgenic strain according to claim 1, wherein expressions of the native genes encoding GlgC and SdhB are both suppressed in the host cell.

3. The transgenic strain according to claim 1, further comprising a recombinant plasmid in the host cell, wherein the recombinant plasmid comprises the first exogenous gene, the second exogenous gene, the third exogenous gene, and the fourth exogenous gene.

4. The transgenic strain according to claim 1, wherein the first exogenous gene, the second exogenous gene, the third exogenous gene, and the fourth exogenous gene are interposed in the genome of the host cell.

5. The transgenic strain according to claim 1, wherein the host cell is selected from the group consisting of Synechococcus elongatus PCC7942, Synechococcus elongatus UTEX2973, Synechocystis sp.PCC6803, and Synechococcus sp.PCC7002.

6. The transgenic strain according to claim 1, wherein the host cell is a cyanobacterium comprising the native genes encoding GlgC, SdhA, and SdhB, and capable of producing phosphoenolpyruvate with carbon dioxide.

7. The transgenic strain according to claim 1, wherein the transgenic strain is capable of consuming carbon dioxide to produce succinate under the condition of providing carbon dioxide and light, and excreting the produced succinate out of the host cell.

8. The transgenic strain according to claim 1, wherein knockdown of at least one of the genes encoding GlgC, SdhA, and SdhB in the transgenic strain is performed by CRISPRi for gene suppression.

9. The transgenic strain according to claim 1, wherein the exogenous genes further comprises a gene encoding a succinate transport protein.

10. The transgenic strain according to claim 1, wherein the exogenous genes further comprises genes encoding D-xylose-proton symporter (xylE), D-xylose dehydrogenase (xylB), D-xylono-1,5-lactone lactonase (xylC), one of xylonate dehydratase (yjhG) or xylanase D (xylD), 2-keto-3-deoxyxylonate dehydratase (xylX), and α-ketoglutaric semialdehyde dehydrogenase (xylA).

11. The transgenic strain according to claim 10, wherein the transgenic strain is capable of consuming xylose to produce succinate in the presence of xylose, and excreting the produced succinate out of the host cell.

12. A method for producing succinate, comprising:
a) providing a transgenic strain according claim 1; and
b) culturing the transgenic strain under a first preset condition,
wherein the first preset condition comprises providing light and providing carbon dioxide.

13. A method for producing succinate, comprising:
a) providing a transgenic strain according to claim 10; and
b) culturing the transgenic strain under a first preset condition, a second preset condition, and a combination thereof,
wherein the first preset condition comprises providing light and providing carbon dioxide;
and the second preset condition comprises providing xylose.

14. The method according to claim 13, wherein b) further comprises performing the first preset condition and the second preset condition alternately.

15. The method according to claim 12, wherein the first preset condition comprises providing light at a light intensity of 100-800 uE.

16. The method according to claim 15, wherein the first preset condition comprises providing light at a light intensity of 200 uE.

17. The method according to claim 15, wherein the first preset condition further comprises: introducing mixed air having a gas composition comprising 0 to 5% carbon dioxide at a flow rate of 20 to 80 cc/min to a culture medium in which the transgenic strain is cultured at a culture temperature of 30° C.
